(19) United States
(12) Patent Application Publication
(10) Pub. No.: US 2005/0215899 A1
(43) Pub. Date: Sep. 29, 2005

(54) Methods, systems, and computer program products for acoustic radiation force impulse (ARFI) imaging of ablated tissue

(76) Inventors: Gregg E. Trahey, Hillsborough, NC (US); Kathryn R. Nightingale, Durham, NC (US); Stephen W. Smith, Durham, NC (US); Brian J. Fahey, Durham, NC (US)

Correspondence Address: MYERS BIGEL SIBLEY & SAJOVEC, PO BOX 37428, RALEIGH, NC, 27627, US

(21) Appl. No.: 11/037,539

(22) Filed: Jan. 18, 2005

Related U.S. Application Data
(60) Provisional application No. 60/536,782, filed on Jan. 16, 2004.
(60) Provisional application No. 60/537,134, filed on Jan. 16, 2004.
(60) Provisional application No. 60/536,783, filed on Jan. 15, 2004.

(51) Int. Cl. A61B 8/00; A61B 8/12; A61B 8/14

(52) U.S. Cl. 600/439

(57) ABSTRACT

Ultrasound methods of distinguishing ablated tissue from unablated tissue include scanning ablated tissue using Acoustic Radiation Force Impulse (ARFI) imaging. ARFI image data is generated based on the scanning. The image data includes a portion of increased stiffness representing the ablated tissue that is distinguishable from unablated tissue.

RELATED APPLICATIONS

This application claims the benefit of U.S. Provisional Application Ser. Nos. 60/536,782, filed Jan. 15, 2004; 60/537,134, filed Jan. 16, 2004; and 60/536,783, filed Jan. 15, 2004, the disclosures of which are incorporated by reference herein in their entirety.

### FIELD OF THE INVENTION

The present invention relates to ultrasound methods and apparatus for the identification and/or characterization of regions of altered stiffness in a target media, and more particularly, for the identification and/or characterization of ablated tissue.

### BACKGROUND OF THE INVENTION

Ablation therapy is a minimally-invasive clinical treatment in which target cells are destroyed via the introduction of localized extreme temperatures. Capable of being implemented through several different techniques, including cryosurgical, radiofrequency (RF), high intensity focused ultrasound (HIFU) methods, microwave and laser techniques, ablation procedures have become popular choices in the treatment of many soft-tissue cancers and cardiac arrhythmias. Vital to the success of any ablation procedure is the ability to precisely control lesion size. The induced lesion must be of adequate volume to completely destroy the target cancer or completely isolate the aberrant cardiac pathway. However, in order to minimize damage to surrounding healthy tissues, lesions should not be excessively large.

Several imaging modalities, including intracardiac echocardiography (ICE), conventional sonography, magnetic resonance imaging (MRI), and elastography, have been utilized in attempts to monitor ablation procedures. Sonography may not perform well in characterizing lesion size or boundaries during RF- or HIFU-based tissue ablations. In some B-Mode images, a hyper echoic region may be present post ablation that corresponds to gas bubbles formed during tissue vaporization. However, lesions are often formed without bubble creation, and, even when visible, these bubbles may not allow for lesion characterization with any degree of precision. Elastography and MRI have been used in the imaging of ablation lesions.

### SUMMARY OF THE INVENTION

According to embodiments of the present invention, ultrasound methods of distinguishing ablated tissue from unablated tissue include scanning ablated tissue using Acoustic Radiation Force Impulse (ARFI) imaging. ARFI image data is generated based on the scanning. The image data includes a portion of increased stiffness representing the ablated tissue that is distinguishable from unablated tissue.

According to some embodiments, an ultrasound system for distinguishing ablated tissue from unablated tissue includes an ultrasound transducer array configured to scan ablated tissue using Acoustic Radiation Force Impulse (ARFI) imaging. A processor is configured to generate ARFI image data based on the scanning. The image data includes a portion of increased stiffness representing the ablated tissue that is distinguishable from unablated tissue.

According to further embodiments of the invention, methods of ablating tissue include ablating a portion of the tissue and scanning the tissue using Acoustic Radiation Force Impulse (ARFI) imaging to provide an ARFI image of the ablated portion of the tissue. Characteristics of the ablated portion of the tissue are identified using the ARFI image.

### BRIEF DESCRIPTION OF THE DRAWINGS

FIGS. 1A-1B are block diagrams of system according to some embodiments of the present invention.

FIG. 2 illustrates the sequential scanning of the two dimensional plane of target regions of FIG. 1B, with different forced regions in each cycle, to produce a two dimensional displacement map for each forced region.

FIG. 3 illustrates the signal processing operations implemented by the signal processing device (31) of FIG. 1, in which the two dimensional displacement maps for each forced region, as generated in FIG. 2, are combined into a single image.

FIGS. 4A-4B are flowcharts illustrating operations according to embodiments of the present invention.

FIG. 5 illustrates embodiments of an orientation of a transducer, and the terminology associated with the different dimensions: axial, azimuthal, and elevation.

FIG. 6A illustrates embodiments of generating a three-dimensional volume using a translation stage connected to the transducer, which allows the interrogation of multiple axial/azimuthal planes by translating the transducer in the elevation dimension.

FIG. 6B illustrates embodiments of a two-dimensional transducer (which has several rows of elements) to interrogate a three-dimensional volume. In this approach, the transducer is held stationary, and the different axial/azimuthal planes are interrogated via electronic focusing.

FIGS. 7A-7D are B-Mode and ARFI images of the left ventricle before and after cardiac ablation. The scale for the ARFI images is displacement in pm. FIG. 7A is a reference B-Mode image, FIG. 7B is a reference ARFI image, FIG. 7C is a B-Mode image after one 60 second ablation, and FIG. 7D is an ARFI image after one 60 second ablation.

FIGS. 8A-8J are B-Mode and ARFI images of the left ventricle before, during and after cardiac ablation. The scale for ARFI images is displacement in pm. Arrows indicate the location of catheter-tissue interface when visible in the B-Mode images. FIG. 8A is a reference B-Mode image, FIG. 8B is a reference ARFI image, FIG. 8C is a B-Mode image 14 seconds into the ablation procedure, FIG. 8D is an ARFI image 14 seconds into the ablation procedure, FIG. 8E is a B-Mode image 28 seconds into the ablation procedure, FIG. 8F is an ARFI image 28 seconds into the ablation procedure, FIG. 8G is a B-Mode image 42 seconds into the ablation procedure, FIG. 8H is an ARFI image 42 seconds into the ablation procedure, FIG. 8I is a B-Mode image after one 60 second ablation procedure, and FIG. 8J is an ARFI image after one 60 second ablation procedure.

FIG. 9 is an RF ablation lesion (outlined in white) in the left ventricle of a sheep heart. The photograph was taken after several ablation procedures of various durations were performed.

FIGS. 10A-10B are time-gain compensated ARFI images of in vivo cardiac ablations in sheep. Darker regions correspond to regions of smaller displacement. FIG. 10A is the time-gain compensated image of FIG. 7D and FIG. 10B is the time-gain compensated image of FIG. 8J.

FIGS. 11A-11D are B-Mode and an ARFI M-Mode images of a sheep left ventricle. The B-Mode image is shown in FIG. 11A. FIGS. 11B-11D are the ARFI M-Mode images of the left, center, and right target regions of tissue, respectively. Arrows in FIG. 11A shown the target lines of flight for the ARFI M-Mode investigations. Each region of tissue was investigated for 0.64 seconds.

FIGS. 12A-12D are B-Mode and ARFI images of a liver sample before and after RF ablation procedures. The ARFI scale on the right of FIGS. 12B and 12D corresponds to displacement in μm. FIGS. 12A and 12B are reference images for B-Mode and ARFI images, respectively. FIGS. 12C and 12D are B-Mode and ARFI images, respectively, after two 60 second RF ablations.

FIGS. 13A-13B are time-gain compensated ARFI images corresponding to FIGS. 13A-13B correspond to FIGS. 12A-12B, respectively. FIG. 13A is a pre-ablation image, and FIG. 13B is a post-ablation image.

FIG. 14 is a thermal lesion in a bovine liver sample created by RF ablation.

FIGS. 15A-15D are B-Mode and ARFI images of the left ventricle before and after cardiac ablation. The ARFI displacement scale on the right of FIGS. 15B and 15D correspond to displacement in μm.

FIGS. 16A-16F are B-Mode and ARFI images of the left ventricle before, during and after cardiac ablation. The ARFI displacement scale on the right of FIGS. 16B, 16D and 16F correspond to displacement in μm. FIGS. 16A and 16B correspond to reference B-Mode and ARFI images, respectively. FIGS. 16C and 16D correspond to B-Mode and ARFI images, respectively, 28 seconds into ablation. FIGS. 16E and 16F correspond to B-Mode and ARFI images, respectively, after one 60 second ablation.

FIGS. 17A and 17B are time-gain compensated ARFI images of invivo cardiac ablations in sheep. TGC processing allows for stiffness comparisons to be made over larger axial spans. The intensity scale of the ARFI images does not correspond to actual tissue displacements. FIG. 17A corresponds to FIG. 14D and FIG. 17B corresponds to FIG. 16D.

FIGS. 18A-18F are B-Mode and ARFI images of liver sample before and after formaldehyde injection. The scale for ARFI images is displacement in Jm. FIGS. 18A and 18B show reference B-Mode and ARFI images, respectively. FIGS. 18C and 18D show images acquired 2 min after formaldehyde injection, while FIGS. 18E and 18F show images acquired 10 min after formaldehyde injection. Arrows in FIGS. 18C and 18E refer to hyperechoic regions due to presence of formaldehyde.

FIG. 19 is a photograph of formaldehyde-induced lesion (outlined in white) in bovine liver sample. During data acquisition, the portion of liver in the bottom of photograph was closest to face of the transducer. The photograph was taken 15 minutes after formaldehyde injection. The scale of the ruler is in cm.

FIGS. 20A-B are ARFI images of lesion growth over two different 4 min intervals. FIG. 20A shows lesion growth during first 4 min of formaldehyde exposure, and FIG. 20B shows lesion growth from 6 min to 10 min after formaldehyde injection. In order to show smaller increases in lesion size clearly, FIG. 20B is shown with one-third the dynamic range as FIG. 20A. Images created by subtracting displacement values of later (in time) data sets from earlier data sets. Hence, positively-valued areas (light-gray and white) denote tissue regions that experienced increased stiffness (i.e. lesion formation). The scale of the images is displacement differences in μm.

FIGS. 21A-21F are B-Mode and ARFI images of liver sample before and after RF ablation procedure. FIGS. 21A and 21B show reference B-Mode and ARFI images, respectively, acquired before the ablation procedure. FIGS. 21C and 21D show images acquired after one 60 s ablation procedure was performed, and FIGS. 21E and 21F show images acquired after two 60 s ablation procedures had been performed. The scale for the ARFI images is displacement in μm. The arrow in FIG. 21E refers to the hyperechoic region caused by the formation of gas bubbles.

FIG. 22 is a photograph of thermal lesion (outlined in white) in bovine liver sample imaged in FIGS. 21A-21F. During data acquisition, the portion of the liver in top of photograph was closest to face of the transducer. The scale of the ruler is in cm.

FIGS. 23A-23D are B-Mode and ARFI images of a liver sample before and after a lower energy RF ablation procedure. FIGS. 23A and 23B show reference B-Mode and ARFI images, respectively, acquired before the ablation procedure. FIGS. 23C and 23D show images acquired after one 40 s ablation procedure was performed. The scale for the ARFI images is displacement in μm.

FIGS. 24A-24C illustrate images of an ex vivo bovine liver sample after HIFU ablation. FIG. 24A is a conventional B-Mode ultrasound image, FIG. 24B is an ARFI displacement image, and FIG. 24C is a pathology image of the ex vivo bovine liver sample after HIFU ablation. The scale for FIG. 24B is displacement in μm, and the scale of the ruler in FIG. 24C is in cm.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. It will also be appreciated by those of skill in the art that references to a structure or feature that is disposed “adjacent” another feature may have portions that overlap or underlie the adjacent feature.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer (such as an ultrasound device), and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the invention may be carried out on human subjects for diagnostic or prognostic purposes, and may be carried out on animal subjects such as dogs and cats for veterinary purposes.

Numerous variations and implementations of the instant invention will be apparent to those skilled in the art. Ultrasound apparatus is known, and is described in, for example, U.S. Pat. No. 5,487,387 to Trahey et al.; U.S. Pat. No. 5,810,731 to Sarvazyan and Rudenko; U.S. Pat. No. 5,921,928 to Greenleaf et al.; M. Fatemi and J. Greenleaf, Ultrasound-stimulated vibro-acoustic spectrography, Science, 280:82-85, (1998); K. Nightingale, Ultrasonic Generation and Detection of Acoustic Streaming to Differentiate Between Fluid-Filled and Solid Lesions in the Breast, Ph.D. thesis, Duke University, 1997; K. Nightingale, R. Nightingale, T. Hall, and G. Trahey, The use of radiation force induced tissue displacements to image stiffness: a feasibility study, 23^{rd} International Symposium on Ultrasonic Imaging and Tissue Characterization, May 27-29, 1998; K. R. Nightingale, P. J. Kornguth, S. M. Breit, S. N. Liu, and G. E. Trahey, Utilization of acoustic streaming to classify breast lesions in vivo, In Proceedings of the 1997 IEEE Ultrasonics Symposium, pages 1419-1422, 1997; K. R. Nightingale, R. W. Nightingale, M. L. Palmeri, and G. E. Trahey, Finite element analysis of radiation force induced tissue motion with experimental validation, In Proceedings of the 1999 IEEE Ultrasonics Symposium, page in press, 1999; A. Sarvazyan, O. Rudenko, S. Swanson, J. Fowlkes, and S. Emelianov, Shear wave elasticity imaging: A new ultrasonic technology of medical diagnostics, Ultrasound Med. Biol. 24:9 1419-1435 (1998); T. Sugimoto, S. Ueha, and K. Itoh, Tissue hardness measurement using the radiation force of focused ultrasound, In Proceedings of the 1990 Ultrasonics Symposium, pages 1377-1380, 1990; and W. Walker, Internal deformation of a uniform elastic solid by acoustic radiation force, J. Acoust. Soc. Am., 105:4 2508-2518 (1999). The disclosures of these references are to be incorporated herein by reference in their entirety for their teaching of various elements and features that may be used to implement and carry out the invention described herein.

Although embodiments according to the invention are described herein with respect to examples of ARFI imaging of organs such as hearts and livers, it should be understood that the present invention may include ARFI imaging of other organs, and in particular, of soft tissue organs having ablated tissue. According to embodiments of the present invention, ultrasound transducers configured for ARFI imaging may be positioned on or inside of organs, for example, using ultrasound transducers carried by delivery devices, such as catheters or endoscopes. Ultrasound transducers may be positioned inside body cavities, blood vessels and/or ducts, on body tissue or organs, or externally to the patient. In accordance with embodiments of the invention, in vivo and/or ex vivo ARFI imaging may be performed.

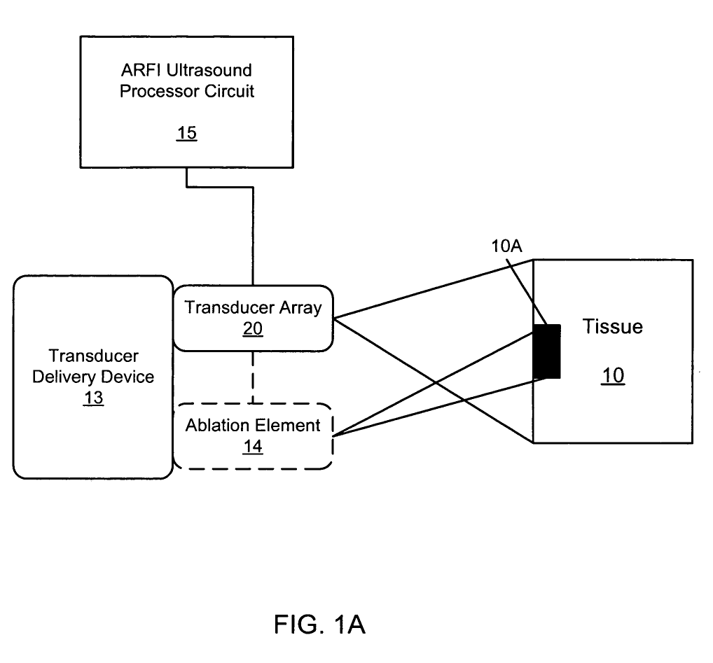

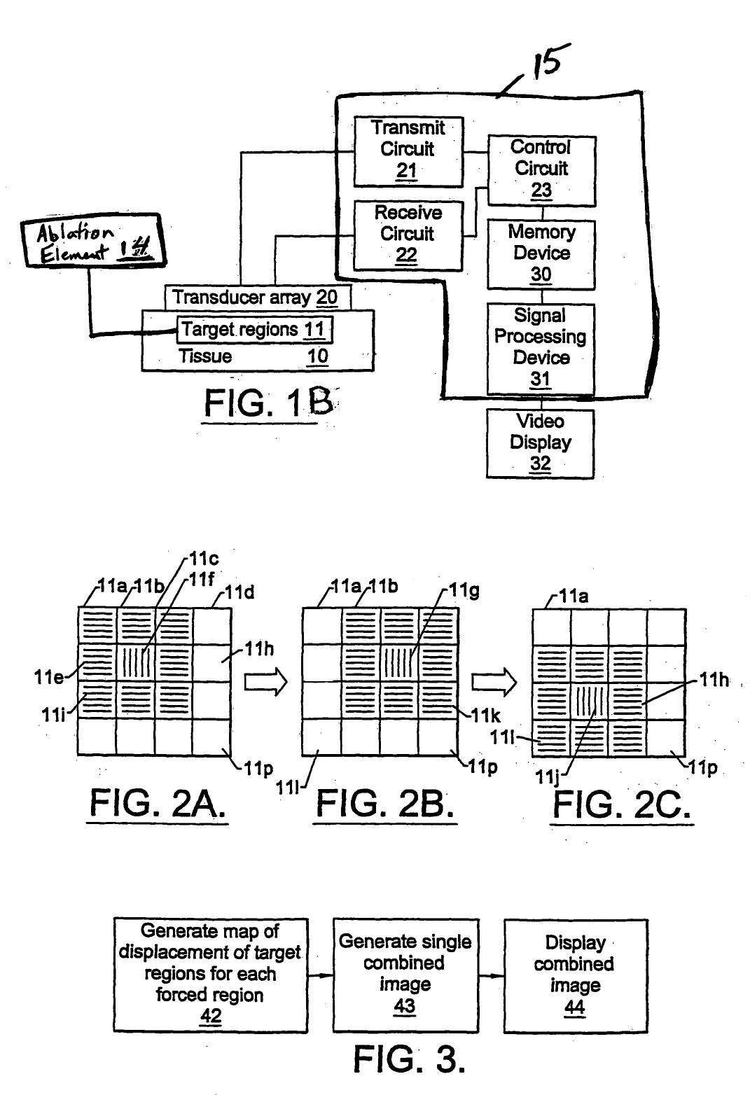

FIGS. 1A-1B illustrate ultrasound systems according to embodiments of the invention. The system (or apparatus) includes a transducer array 20 on a transducer delivery device 13 (such as a catheter, endoscope or the like), an Acoustic Radiation Force Impulse (ARFI) ultrasound processor circuit 15, and an optional ablation element 14. As shown in FIG. 1A, a region of tissue 10 that is interrogated by the transducer array 20 includes ablated tissue 10A. The processor circuit 15 and the transducer array 20 are configured to scan the ablated tissue 10A using ARFI imaging. The processor circuit 15 is configured to generate ARFI image data based on the scanning. The image data includes a portion thereof that corresponds to a region of increased stiffiess representing the ablated tissue 10A that is distinguishable from image data corresponding to unablated tissue regions in the tissue 10.

Acoustic Radiation Force Impulse (ARFI) imaging generally refers to ultrasound techniques using both relatively high energy “pushing” pulses that can induce a physical displacement of the tissue and relatively low energy “tracking” pulses. Examples of ARFI imaging techniques are described herein and in U.S. Pat. No. 6,371,912 to Nightingale, the disclosure of which is hereby incorporated by reference in its entirety.

The ablated tissue 10A may be ablated by the optional ablation element 14, or the ablated tissue 10A may be ablated by an ablation element provided as part of another device. The ablation element 14 may be controlled by the processor circuit 15, or a separate controlling circuit may be provided. The ablation element 14 can be any suitable ablation element, including ablation elements configured to ablate tissue using cryosurgical, radiofrequency (RF), chemical, high intensity focused ultrasound (HIFU) methods, microwave and/or laser techniques. In some embodiments, the ablation element 14 is a separate ultrasonic ablation device, such as a HIFU transducer. However, the ablation element 14 may be provided as part of the transducer array 20. For example, the same transducer elements of the transducer array 20 may be used to both ultrasonically ablate the tissue and to perform ARFI and/or conventional B-mode ultrasound imaging. As another example, the transducer array 20 can include some transducer elements configured to ultrasonically ablate tissue and other transducer elements configured for ARFI and/or B-mode ultrasound imaging.

In the configuration illustrated, for example, in FIG. 1A, characteristics of the ablated tissue 10A can be identified based on the ARFI image data from the processor circuit 15. For example, the size and/or the position of the ablated tissue 10A can be identified from the ARFI image data, for example, by displaying the image data on a display. The ARFI image data can be used in substantially real-time to monitor the ablation of the ablated tissue 10A. For example, the ablated tissue 10A can be scanned by the transducer array 20 repeatedly to monitor the size and/or shape of the ablated tissue 10A during ablation. That is, ablation and ARFI ultrasound scanning may be performed at the simultaneously or ablation and ARFI ultrasound scanning may be performed repeatedly in alternating steps to monitor the ablated tissue.

The ARFI ultrasound imaging data can be used to provide a two- or a three-dimensional image.

In some embodiments, the ablation element 14 is omitted. The ablated tissue 10A can be a region of tissue 10 that is ablated in a procedure prior to an ARFI ultrasound scan. In some cases, it may be advantageous to monitor the ablated tissue 10A over time to determine if a change has occurred. For example, if the ablated tissue 10A heals over time, another ablation procedure may be desired. Changes in the size, the shape and/or the position of the ablated tissue 10A may be determined based on comparing the ARFI images taken at different times. In some embodiments whether or not the ablation element is not omitted, the ARFI image can be utilized during and or after the ablation procedure to ascertain if all of the target tissue (i.e. cancer or arrhythmogenic tissue) has been successfully destroyed.

The transducer array 20 and/or the ablation element 14 can be mounted on the delivery device 13. The delivery device 13 can be a device configured to internally scan tissue in vivo, such as a catheter or endoscope. In some embodiments, the ablation element 14 and the transducer array 20 are mounted on the same catheter or endoscope. However, it should be understood that external scanning may also be performed.

The ultrasound transducer array 20 is configured to provide ARFI imaging data, and may be a one-dimensional array or a two-dimensional array. As illustrated in FIG. 1B, the array 20 is directed to a two-dimensional plane comprising one or more target regions 11 within the tissue 10. A transmit circuit 21 is operatively associated with the transducer array and delivers high energy “pushing” pulses to a forcing region among the target regions (i.e., pulses that can induce a physical displacement of the tissue within the target regions), as well as for delivering relatively lower energy “tracking” pulses. A receive circuit 22 is connected to the transducer array 20 to receive information from the target regions 11 for subsequent signal processing. The transmit circuit 21 and detector circuit 22 are both operatively associated with an appropriate control circuit 23 that triggers the pushing pulses and tracking pulses, organizing information received from the target regions for subsequent signal processing, and which also cycles the pushing pulses and corresponding tracking pulses through different forcing regions.

Information received by receive circuit 22 can be stored in a memory device 30 such as a random access memory or other suitable memory device, which can store both initial and displaced positions of target regions. A signal processing device or signal processor 31 is operatively associated with the memory device 30, and serves as a means for generating initial images for particular forced regions and a single combined image for a plurality of forced regions.

ARFI imaging can include one or more of the following:

(a) delivering a set of tracking pulses from a plurality of transducer elements in an ultrasound transducer array to one or a plurality of target regions in a two-dimensional plane within the medium to detect an initial positions for the one or plurality of target regions;

(b) storing the echoes that reflect the initial positions for the one or plurality of target regions; then

(c) delivering a first set of pushing pulses from the plurality of transducer elements to a forcing region among the target regions to displace the target regions to subsequent (e.g., displaced) positions;

(d) delivering a second set of tracking pulses from the plurality of transducer elements in the ultrasound transducer array to the one or plurality of target regions to detect subsequent positions for the one or plurality of target regions,

(e) storing the echoes that reflect the displaced positions for the one or plurality of target regions;

(f) repeating steps (a) through (e) in a series of cycles, with the pushing and tracking pulses being delivered from a different plurality of transducer elements or the same plurality of transducer elements in the array to a different forcing region, and optionally to a plurality of different target regions, during each of the cycles;

(g) generating a two-dimensional displacement map from each of the initial positions and displaced positions for each of the forcing regions to produce a plurality of two-dimensional displacement maps; and then

(h) combining the plurality of two-dimensional displacement maps into a single combined image, with a region of increased stiffness being indicated by a region of decreased displacement within the combined image, or a region of decreased stiffness being indicated by a region of increased displacement within the combined image.

Step (d) above may optionally be carried out while concurrently delivering an interspersed set of pushing pulses to the forcing region to reduce the return of the target regions from the displaced positions to the initial positions.

Steps (a) through (e) above may be completed in a total of 50, 25 or 10 milliseconds or less for each cycle (i.e., each forced region). A cycle of steps (a) through (d) may be completed in 15 milliseconds or less.

In some embodiments, the pushing pulses are delivered before the first set of tracking pulses, the initial positions are displaced positions, and the second positions are relaxed positions. In another embodiment, the pushing pulses are delivered between the first and second set of pulses, the initial positions indicate the relaxed positions, and the second positions indicate the displaced positions.

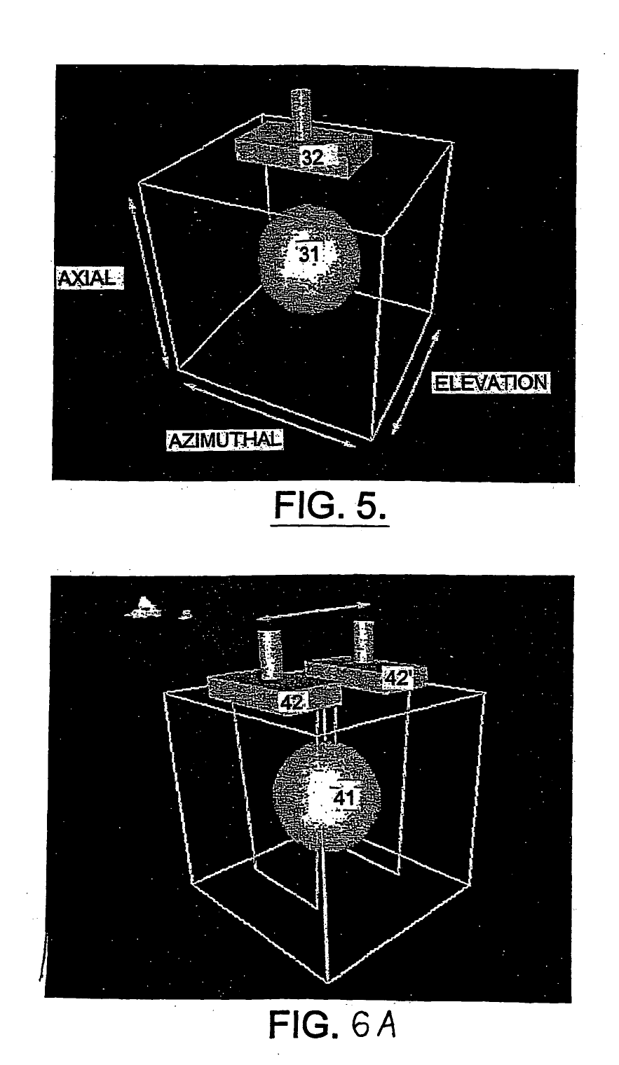

FIG. 2 illustrates the cyclic repeating of steps (a) through (e) above for different forced regions (vertical hatched regions 11f, 11g, and 11j) within the target regions (11a through 11p) in the axial/azimuthal plane (see FIG. 5). The Boxes represent the same view as that shown in FIG. 1B. Arrows represent transition from one cycle to another (cycles A, B, and C). Note that not all target regions need be detected during each cycle, and hence the corresponding transducer elements may be active or inactive in various patterns during each cycle.

As shown in Block 42 of FIG. 3, a two-dimensional displacement map can then be generated for each cycle A, B, and C of FIG. 2. These two-dimensional displacement maps are then used to generate a single combined image (Block 43) in the signal processing device 31 of FIG. 1B. This combined image can then be displayed (Block 44) on the video display device 32 of FIG. 1. Of course, the single combined image may also be stored in a suitable memory device for future reference, printed on a printer, etc. A B-mode image of the two dimensional plane in accordance with conventional techniques may also be generated, and the single combined image superimposed on that B-mode image may be displayed.

Embodiment according to the invention can be implemented on a Siemens Elegra or Antares ultrasound scanner, modified to provide control of beam sequences and access to raw radio frequency data. Siemens 75L40, VF105, VF73, CH62, AcuNav, and similar transducers may be used as the transducer array.

Particular embodiments of the invention may be carried out as follows:

First, a group of low intensity “tracking lines” that interrogate the tissue surrounding the position of interest are fired and stored for tissue initial position reference.

Second, a series of one or more focused, high intensity “pushing lines” is fired along a single line of flight focused at the position of interest.

Third, the original group of tracking lines is fired again, in order to determine the relative motion caused by the radiation force associated with the pushing lines. These tracking lines may optionally be interspersed with pushing lines in order to reduce or avoid relaxation of the tissue.

Fourth, each tracking line is divided into sequential axial search regions, and the displacements of the tissue within each search region are determined. A number of different motion tracking algorithms can be used to determine the relative motion, or displacement, between the initial reference tracking lines and the second set of tracking lines fired after radiation force application. Examples include, but are not limited to, cross correlation and Sum Absolute Difference (SAD). The a priori knowledge of the direction of motion reduces the algorithm implementation time.

Steps 1-4 above may be accomplished in 50, 25 or 10 milliseconds or less. The results of step 4 are used to generate a two-dimensional displacement map of the region of tissue surrounding the position of interest (or force location).

Fifth, steps 1 through 4 can be repeated, cyclically, for a plurality of force locations within a larger two-dimensional imaging plane. The number of forcing locations and the spatial distribution of the forcing locations may be determined by (among other things) the specific transducer, transmit parameters, and the size of the region of interest to be interrogated. The same or different sets of elements within the transducer array may be used for the tracking pulses with each force location.

Sixth, each of the two dimensional displacement maps (each of which may be generated before, during or after subsequent cyclical repeatings of steps 1-4) can be combined into a single image (which may or may not be displayed on a video monitor, printer or other such display means). Signal processing such as averaging of collocated regions, and/or some type of normalization to account for the displacement generated in a homogeneous region of tissue, may be employed.

Note that it is also possible with certain embodiments of the invention to monitor the displacement of the tissue over time, both while the force is being applied (by interspersing the pushing lines and the tracking lines), and after cessation of the high intensity pushing lines or pulses. This is accomplished by firing the group of tracking lines repeatedly at the desired time intervals, and evaluating the changes in the displacement maps over time.

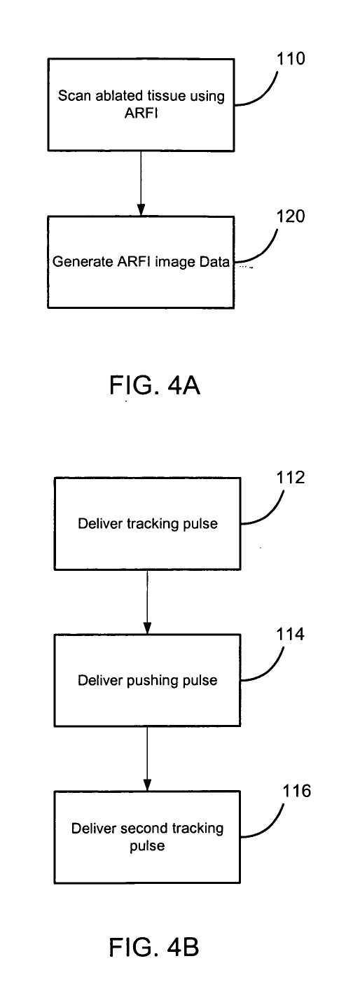

With reference to FIG. 4A, according to embodiments of the present invention, ablated tissue is scanned using ARFI imaging, for example, using techniques described herein (Block 110). ARFI image data is generated based on the scanning (Block 120). The image data includes a portion thereof that corresponds to a region of increased stiffness representing the ablated tissue that is distinguishable from the another portion of the image data corresponding to unablated tissue. The ablated tissue can be identified or characterized based on the image data. As illustrated in FIG. 4B, scanning the ablated tissue can include the following: A tracking pulse is delivered from an ultrasound transducer array to the tissue to detect an initial position for the tissue (Block 112); a pushing pulse is delivered from the ultrasound transducer array to the tissue to displace the tissue to a displaced position (Block 114); and a second tracking pulse from the ultrasound transducer array can be delivered to the disuse to detect the displaced position of the tissue (Block 116).

It has been observed that some tissues exhibit strain-stiffening behavior (e.g., ablated tissue) whereas other tissues do not (e.g., nonablated tissue). Therefore, in methods intended to characterize the stiffness of tissue, it is often advantageous to pre-compress the tissue. This has the effect of increasing the contrast between the different tissue types (Krouskop et. al., Elastic Moduli of Breast and Prostate Tissues Under Compression, Ultrasonic Imaging 20, 260-274 (1998)).

For clarity, the interrogation of a two-dimensional plane with multiple pushing locations (the axial/azimuthal plane-see FIG. 5 where transducer array 32 is positioned over a target region represented as a cube containing a region of varying stiffness 31) has been described. In other embodiments this method is carried out in a manner that includes the interrogation of a three-dimensional volume. This is accomplished in a variety of ways. According to a first example illustrated in FIG. 6A, where a transducer array 42 is positioned over a target region represented as a square containing a region of varying stiffness 41, and is translated from a first position as shown by 42 to a second position shown by 42′, one can use the existing planar system, and translate the transducer in the elevation dimension to sequentially interrogate a series of planes comprising a three-dimensional volume. According to a second example illustrated in FIG. 6B, where transducer array 52 is positioned over a target region represented as a square containing a region of varying stiffness 51, one can use a two-dimensional transducer array (i.e. one that has several rows of elements), and keep the transducer in one location, and steer the beam (represented as lines within the cube) to interrogate a three-dimensional sector of the target region.

When using the ultrasound transducer array to either generate the high intensity pushing pulses, or the displacement tracking pulses, a set of multiple elements may be used to generate each line. The set of elements that is used can either comprise all of the elements in the transducer array, or include only a subset of the elements. The specific elements that are active for each transmit pulse is dictated by the desired focal depth, resolution, and depth of field for each line. According to a particular embodiment, the pushing beams can be tightly focused, therefore a fairly large number of elements can be used to generate each pushing beam.

The spatial peak temporal average intensities required to generate detectable displacements in tissue vary depending upon the tissue acoustic and mechanical characteristics. They can be from 10 W/cm² to 4000 W/cm², with higher intensities being associated with better Signal-to-Noise-Ratios (SNRs). A trade-off exists, however, between increasing acoustic energy deposition and the potential for tissue heating, which should preferably be minimized. The intensities can be comparable to those used for HIFU (High Intensity Focused Ultrasound) imaging (up to 4000 W/cm²); however, the duration of the application in a specific spatial location may be much smaller (up to 15 milliseconds for ARFI, compared to a few seconds for HIFU). Given the short application time in a single location, the required energy should not pose a significant risk to the patient.

The high intensity acoustic energy can be applied by using a series of multiple, relatively short duty cycle pulses (i.e. 40 pulses, each 10 microseconds long, applied over a time period of 10 milliseconds). ARFI can also be accomplished by delivering the same amount of acoustic energy in a much shorter time period using a single long pulse (i.e. 1 pulse, 0.4 milliseconds long). An important issue is delivering the required amount of acoustic energy to the tissue to achieve a given displacement, which can be accomplished using any number of pulsing regimes. One mode of implementation is to use a single, long pulse (i.e. 0.5 milliseconds), to achieve the initial displacement, and then to intersperse some of the shorter duty cycle (i.e. 10 microseconds) high intensity pulses with the tracking pulses to hold the tissue in its displaced location while tracking. This may reduce the amount of time required at each pushing location, and thus reduce the potential for tissue heating, while at the same time still achieving the desired tissue displacements. The use of a single, long pulse may, however, require additional system modifications. It may, for example, require the addition of heat sinking capabilities to the transducer, as well as modification of a standard power supply to allow the generation of longer pulses.

The displacement data from each pushing location can be combined to form a single image. In order to achieve a uniform image, normalization may be useful. There are three features may benefit from normalization: 1) attenuation, 2) pushing function shape and non-uniformity, and 3) time of acquisition of tracking lines. Each of these features may be normalized out of the image, such that an ARFI image of a homogeneous region of tissue will appear uniform.

It should be understood that various configurations of transducers can be provided on catheters that can be used to provide images using Acoustic Radiation Force Imaging (ARFI). For example, in some embodiments according to the invention, a transducer array having any one of or any combination of the following configurations can be provided on an external ultrasound transducer array or an internal array, such as a catheter or endoscope transducer. For example, sector scanning may be used along one or more axis. Rectilinear or curvilinear scanning may be used along one or more axis. Doppler may or may not be used with pulse wave or color flow ultrasound. Elastography vibration may or may not be used. Ultrasound ARFI imaging may also be combined with drug therapy, ablation and/or hyperthermia treatment techniques, for example, to monitor, evaluate and/or characterize the results of treatment. Three dimensional scanning and/or high intensity focused ultrasound (HIFU) may also be used.

Embodiments according to the present invention are discussed below with respect to the following non-limiting examples.

### EXAMPLE 1

### Acoustic Radiation Force Impulse Imaging of Myocardial Radiofrequency Ablation: In Vivo Results

Acoustic Radiation Force Impulse (ARFI) imaging techniques were used to monitor radiofrequency (RF) ablation procedures in in vivo sheep hearts. Additionally, ARFI M-Mode imaging methods were used to interrogate both healthy and ablated regions of myocardial tissue. While induced cardiac lesions were not visualized well in conventional B-Mode images, ARFI images of ablation procedures allowed determination of lesion size, location, and shape through time. ARFI M-Mode images were capable of distinguishing differences in mechanical behavior through the cardiac cycle between healthy and damaged tissue regions. As conventional sonography is often used to guide ablation catheters, ARFI imaging may be a convenient modality for monitoring lesion formation in vivo.

Imaging/Data Acquisition

Experiments were performed with a Siemens Antares scanner (Siemens Medical Solutions USA, Inc., Ultrasound Division, Issaquah, Wash.) that has been modified to provide users with the ability to specify acoustic beam sequences and intensities, as well as access raw radio frequency data. A Siemens VF10-5 linear array was used to acquire data. This array consists of 192 elements, each 5 mm tall and approximately 0.2 mm wide. A fixed-focus acoustic lens is used in the elevation direction, while focusing in the lateral dimension is achieved electronically via the application of appropriate delays to each active element.

Beam sequences during ARFI data collection consisted of both tracking and pushing beams. The tracking beams were standard B-mode pulses (6.67 MHz center frequency, F/1.5 focal configuration, apodized, pulse repetition frequency (PRF) of 10.6 kHz, with a pulse length of 0.3 μs). The system utilizes dynamic focusing in receive such that a constant F/number of 1.5 is maintained. The beamwidth of the tracking beam can be calculated as λ*F/number, or 0.35 mm. The pushing beam aperture was unapodized with a F/1.5 focal configuration, a center frequency of 6.67 MHz, and a pulse length range of 45-75 μs. The shape of the focal region of the pushing beams is oblong (approximately 4 mm axially, and 0.45 mm laterally and in elevation) and fairly complex. Echoes from pushing pulses were not processed.

ARFI images were generated using 72 pushing locations at focal depths of 10 or 15 mm. Pushing locations were separated laterally by a distance of 0.28 mm, resulting in a lateral region of interest (ROI) of 19.9 mm. At each pushing location both tracking and pushing beams were fired along the same line of flight, as in typical A-line interrogation. The first beam fired was a tracking beam used as a reference to record initial tissue position. Next, a pushing beam was fired that generated an impulse of radiation force. Following the pushing beam, 50 tracking beams were fired at a pulse repetition frequency (PRF) of 10.6 kHz to allow for the measurement of the temporal response of the tissue. Each location was imaged for 5.0 ms,allowing for data from all 72 pushing locations to be acquired in 360 ms.

ARFI M-Mode images were produced using ARFI pulse sequences fired repeatedly along the same line of flight. Pulse sequences were similar to those previously described, with the exception being that significantly more tracking beams were utilized. Three-line ARFI M-Mode images were created by alternating target location between three pre-determined regions of tissue. Each target region was investigated for 10 ms, meaning that each tissue region received an impulse of radiation force every 30 ms. System limitations constrained the number of total beams available for use, and thus the entire viewing window for each of the three locations was 0.64 s. Images were constructed by demultiplexing data such that investigations from the same location were grouped together. Each column in the M-Mode images represents the processed RF data from the 6th tracking beam from each investigation, which generally corresponds to the peak tissue displacement in these data sets.

Prior to off-line processing, a 2 ms linear motion filter was applied to raw RF echo data to remove artifacts stemming from cardiac motion. Data was processed by performing 1-D cross-correlation in the axial dimension between sequentially acquired tracking lines. Each tracking line was divided into a series of search regions, and the location of the peak in the cross-correlation function between a 0.25 mm kernel in the first tracking line and a search region in the next tracking line was used to estimate axial tissue displacement in that region. The kernel regions overlapped one another by 75%.

Selected images were processed using time-gain compensation (TGC) techniques in order to smooth focal gains in displacement located in the focal region of the pushing beams. Average displacements were calculated at each axial depth in the data set and were then normalized relative to each other. Raw displacement data at each axial depth was then divided by the appropriate value to apply a laterally-uniform and axially-varying gain. This technique is used to improve contrast in the image, and also reveals details that would otherwise be lost due to strongly spatially-varying brightness. This processing algorithm may preferably be performed immediately following the removal of radiation force, before significant wave propagation commences in the medium.

Experimental Setup and Procedure

Two sheep were used in this study approved by the Institutional Animal Care and Use Committee at Duke University conforming to the Research Animal Use Guidelines of the American Heart Association. Anesthesia was induced and maintained with isoflurane gas (1-5%). After intravenous (IV) access was obtained, the animal was placed on its left side on a water-heated thermal pad. A tracheostomy was performed and the animal was mechanically ventilated with 95-99% oxygen. To prevent rumenal typany, a nasogastric tube was passed into the stomach. A lateral thoracotomy was performed to expose the heart. A femoral arterial line was placed on the left side via a percutaneous puncture. Electrolyte and respirator adjustments were made based on serial electrolyte and arterial blood gas measurements. An IV maintenance fluid with 0.9% sodium chloride was in-fused continuously. Blood pressure, lead II ECG, and temperature were continuously monitored throughout the procedure.

An RF-ablation system (Model 8002, Cardiac Pathways Corporation, Sunnyvale, Calif.) was used to create cardiac lesions. The system utilized a 10 French ablation catheter, which was inserted into the heart via the femoral artery. Lesions were created at various left ventricular locations using system power settings of 12-17 W and ablation durations of 50-70 s. Temperatures at the tissue-catheter interface were not available due to limitations imposed by the ablation system.

Experiments were performed with a hand-held transducer placed directly on the heart of the sheep. During the first trial, B-Mode and ARFI images of regions of interest were acquired both before and after ablations had occurred. During the second trial, B-Mode and ARFI images were acquired before, during, and after ablation procedures. ARFI images were acquired every seven seconds during ablations, with ARFI images from each data set being displayed on a laptop computer adjacent to the operating table within one second of acquisition. During each acquisition, B-Mode data was first obtained in its entirety (taking approximately 15 ms), followed by ARFI data.

ARFI M-Mode investigations were conducted on both healthy and damaged regions of sheep myocardium. Three-line ARFI M-Mode images were created by varying the location of interrogation between three predetermined tissue regions, then demultiplexing the data into images from each location. Each line of the images is the 6th tracking echo (fired 0.5 ms after the pushing pulse), meaning that the complete images show how each tissue region's response to the same impulse of radiation force changes with time during the 0.64 s investigation window.

Upon completion of imaging, the hearts were resected and examined. Lesion sites were exposed, with care being taken to slice tissues in the approximate imaging plane of the ARFI and B-Mode images. Photographs were taken to document lesion size, shape, and location.

Results

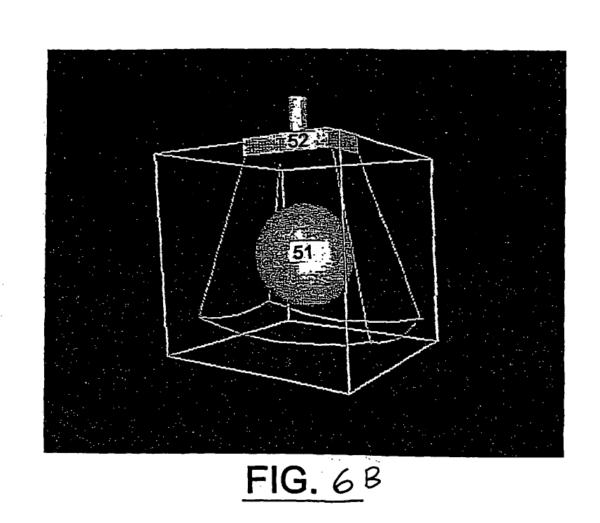

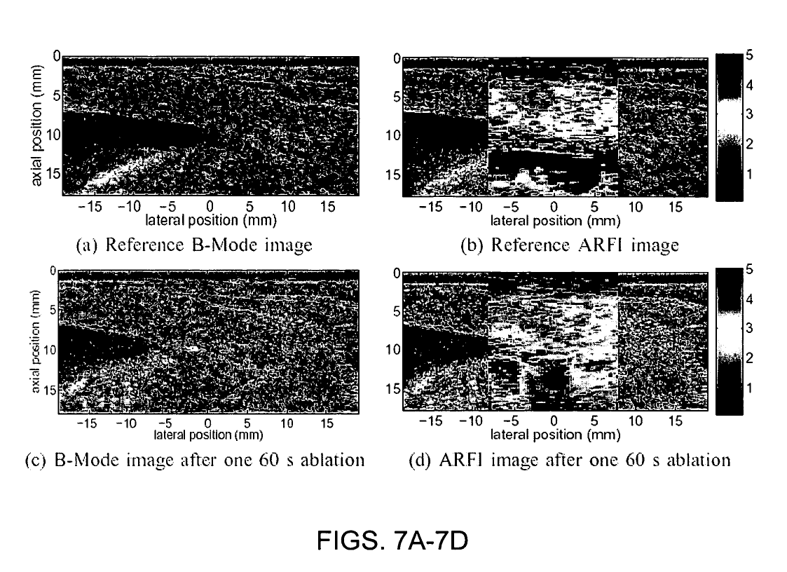

Results from the first trial are presented in FIGS. 7A-7D. FIGS. 7A and 7D show B-Mode images of the apex of the left ventricle before and after ablation, respectively. It is apparent from these images that conventional sonography indicates no change in the cardiac tissue after a lesion has been induced, and that the presence of the lesion cannot be verified in this instance.

FIGS. 7B and 7D show ARFI displacement images which have been centered in the corresponding BMode images for anatomical reference. Stiffer regions are evidenced by smaller displacements (blue). The reference ARFI image, shown in FIG. 7B, demonstrates that the cardiac tissue initially exhibits roughly uniform displacements across the region under investigation (12-17 mm axially). This corresponds to the focal region of the pushing beam, where the applied radiation force is relatively uniform. The regions of smaller displacement (blue) located above 5 mm axially likely arise from a reduced application of radiation force, not necessarily from an increase in tissue stiffness. This focusing issue will be discussed in detail later.

After one 60 s ablation procedure, a second ARFI displacement image was acquired FIG. 7D. Due to cardiac motion, the region of tissue located between 3 and 8 mm laterally in FIG. 7D is now located approximately between −3 and 2 mm laterally. FIGS. 7A-7D demonstrate that the ablated region has experienced an increase in stiffness relative to untreated tissue, and thus the resulting lesion is visualized well utilizing radiation force techniques. The lesion cross-section is circular in shape, with a diameter of approximately 5 mm. The proximal and lateral lesion boundaries are clearly distinguished, and regions of treated and untreated tissue are easily discerned. Although the distal lesion boundary is not easily recognized, its location can be inferred reasonably accurately based upon the curvature of the lateral lesion boundaries. Comparison of ARFI images with their B-Mode companions demonstrates the improvement in lesion visualization that can be achieved by investigating the mechanical properties of tissue. Logistical difficulties prevented the obtainment of a quality imaging plane photograph of the lesion presented in FIGS. 7A-7D.

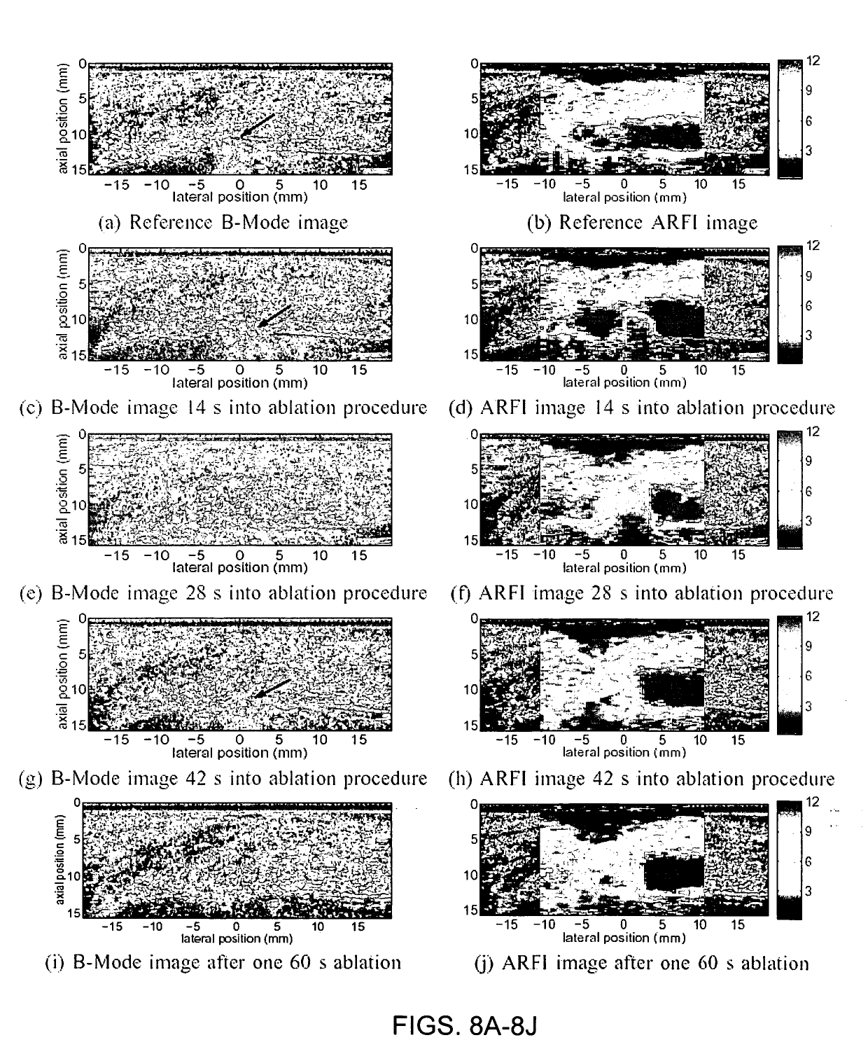

The results from the second sheep trial are shown in FIGS. 8A-8J. In this experiment, B-Mode and ARFI image data was collected before, during, and after ablation of left ventricle myocardium. B-Mode images were acquired prior to ablation (as shown in FIG. 8A), as well as several times during the ablation procedure (FIGS. 8A, 8E, and 8G). The post-ablation B-Mode image is found in FIG. 8I. ARFI displacement images centered in the corresponding B-Mode images are shown in FIGS. 8B, 8D, 8F, 8H, and 8J. The time delay (roughly 15 ms) between B-Mode and ARFI image acquisition prevents complete registration between the two, and thus the B-Mode images provided only an approximated anatomical reference for the corresponding ARFI images.

In the reference B-Mode image shown in FIG. 8A, the position of the ablation catheter tip is observed as a bright region, indicated by the arrow, which casts a shadow into axially deeper regions. The catheter tip is also present in two of the other B-Mode images, while in the remaining two images the catheter tip was positioned outside of the plane of the image. There appears to be minimal, if any, change in the ablation regions shown in the various B-Mode images, and there is certainly not enough information present in any of the images to make precise conclusions concerning lesion size, location, or presence.

In contrast with the B-Mode images, the ARFI displacement images depicted in FIGS. 8A-8J contain valuable information concerning the induced cardiac lesion. As shown in the ARFI reference image FIG. 8B, the ventricle wall is initially fairly uniform in stiffness across the focal region (10-15 mm in depth). During the ablation procedure, the ARFI images show the lesion growing in size over time. In these images (FIGS. 8D, 8F and 8H) the lateral and proximal lesion boundaries are distinct, and lesion size and location is visualized. The distal lesion boundary lies on the endocardium, accounting for the lesion's asymmetric shape as it ends abruptly at the tissue-blood interface. FIG. 8J shows the ARFI image acquired upon completion of the ablation procedure. The lesion has grown to nearly 10 mm in diameter, as shown by its distinct lateral edges. The lack of proximal edge boundary definition stems from the same focusing issue described previously, as the lesion has now grown to the point where its proximal boundary is located a significant distance from the axial focus of the image.

A photograph of the ablated tissue region from the second sheep trial is shown in FIG. 9. The photograph was taken after several additional ablation procedures were performed in the same tissue region as the procedure documented in FIGS. 8A-8J. As a result, the lesion has now grown significantly in size and is visible from the exterior of the heart. However, previous results from our laboratory suggest that ARFI imaging is capable of accurately measuring the size of subdermal lesions created using radiofrequency-based ablation methods.

In FIGS. 7D and 2J, it may be beneficial see the distal and proximal lesion boundaries, respectively, to ascertain a transmural lesion has been created. To that end, time-gain compensation (TGC) techniques, commonly used in conventional B-Mode images, can be implemented, as shown in FIG. 10. As shown, utilizing TGC processing results in improved lesion boundary distinction and a greater appreciation for overall lesion shape.

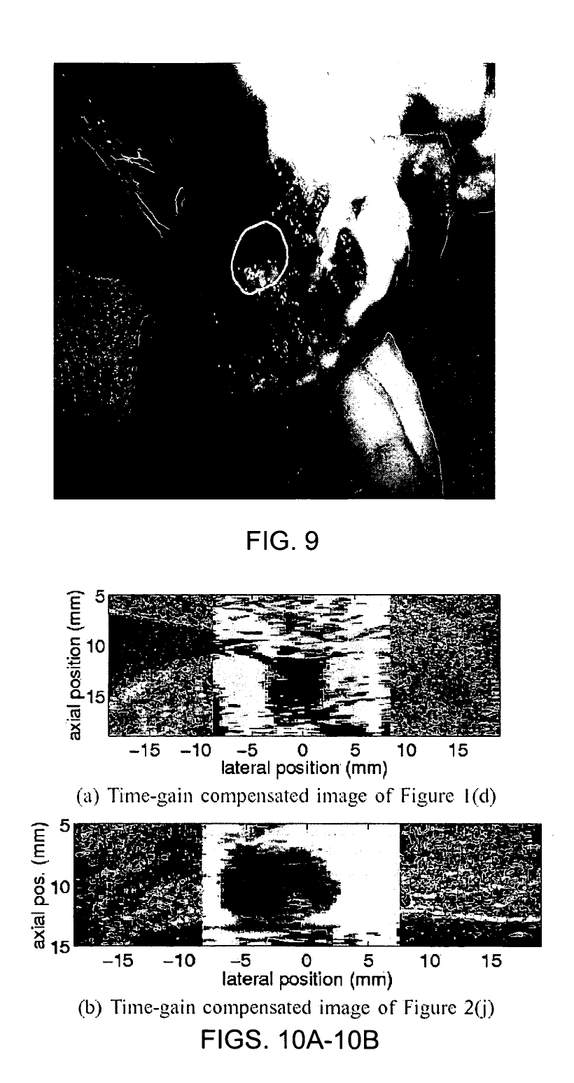

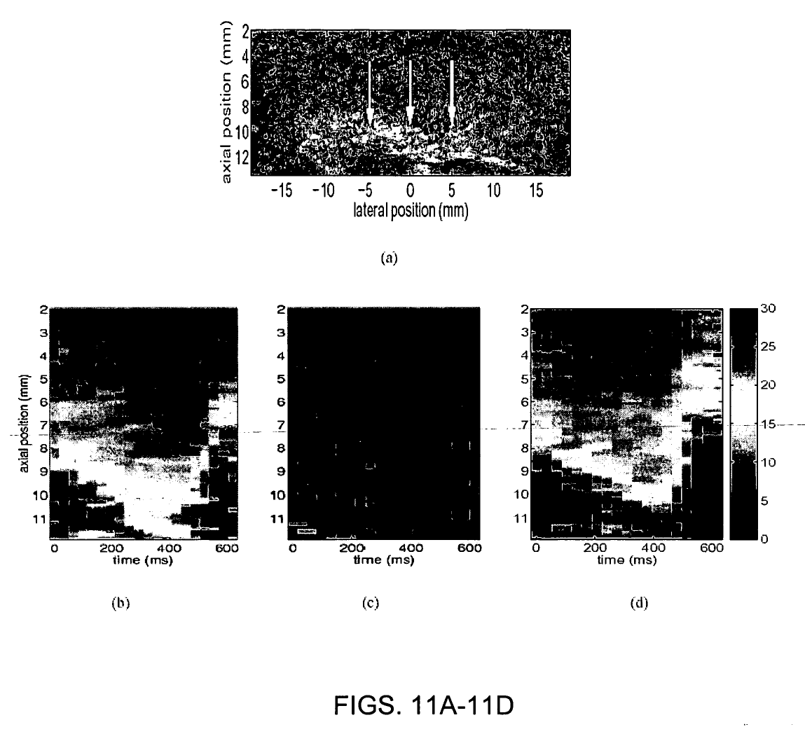

FIGS. 11A-11D show the results from the ARFI M-Mode investigation. The image shown in FIGS. 11B and 11D show M-Mode results from the left and right target regions, respectively, where there existed healthy myocardium. FIG. 11C contains the M-Mode results from the central region of investigation, where a thermal lesion had been created. The corresponding B-Mode image, shown in FIG. 11A, includes arrows indicating the regions of the ventricle wall that were examined. The images indicate that healthy regions of cardiac tissue (FIGS. 11B and 11D respond to impulses of radiation force differently at different times in the cardiac cycle. The ablated region of tissue, however, shows little variation in its response to the applied force through time (FIG. 11C). Although software limitations prevented a precise relationship between lines in the M-Mode image and events in the cardiac cycle from being established, conclusions can be made based upon the fact that each of the three groups of M-Mode investigations displayed in the figure were acquired during the same 0.66 s period of time.

Discussion

It has been demonstrated that ARFI imaging is capable of detecting cardiac tissue mechanical properties in vivo to investigate a beating heart. From the resulting data, anatomical features can be visualized, such as as blood-tissue interfaces, as well as view both the spatial and temporal responses of the myocardium to the applied acoustic radiation force. Although the transducer was placed directly on the heart through an open chest, more clincally-realistic procedures can be developed by using a phased array to image transcutaneously.

The application of high-intensity ultrasound pulses in the frequency range of 1-4 MHz can alter the performance of a frog heart, potentially causing either a premature ventricular contraction or a reduction in aortic pressure. Although aortic pressure was not monitored during our experiments, the ECGs of the animals were monitored continuously. During standard ARFI acquisitions (not during the ablation procedures), observations revealed no arrhythmias in the ECGs of the animals. In addition, during the first trial (when data was acquired only at times when ablation procedures were not being performed) no arrhythmias were apparent in the animal's ECG. Although the use of ARFI on a living sheep heart appeared to be safe during our experiments, further investigation into the safety of ARFI imaging of cardiac tissue may be needed.

The results presented demonstrate that although conventional sonography failed to visualize induced cardiac lesions, ARFI imaging appears to be a promising modality for monitoring cardiac RF ablation therapy in vivo. The ability of ARFI imaging to distinguish thermal lesions effectively stems from the large increase in their elastic modulus relative to untreated tissue. Assuming a uniform distribution of radiation force, stiffer lesions will displace less than healthy tissues. (This is verified by the results in FIGS. 7A-7D and FIGS. 8A-8J, where lesions appear as regions of small displacement (on the order of 1-2 μm), while untreated myocardium tends to be displaced significantly further (typically <5 μm).) However, it is unlikely that radiation force distribution is uniform, as the attenuation of tissue may be permanently increased when tissue temperatures are raised above 40° C. The applied radiation force is proportional to the tissue attenuation coefficient, and in reality, a stronger radiation force may be applied to the lesion than is applied to healthy tissue. This implies that changes in elastic moduli of lesions may be even greater than as illustrated in ARFI displacement images.

As demonstrated in FIGS. 11A-11D, ARFI M-Mode investigations are capable of identifying regions of cardiac tissue that do not exhibit cyclical changes in stiffness. This may be beneficial clinically to assess or diagnose potential heart conditions. For instance, ischemic and infarcted myocardium have been shown to have an increased stiffness relative to healthy tissue, especially in the first one to two weeks after being damaged. ARFI M-Mode imaging could thus potentially be used to image infarcted or ischemic tissues.

One of the challenges involved with utilizing ARFI imaging to monitor lesion formation in vivo is the motion associated with the beating of the heart. During systole and diastole, the lesion being monitored may move in and out of the imaging plane of the transducer. However, as demonstrated by FIGS. 8A-8J, rapid acquisition of many ARFI data sets (in this case once every seven seconds) increases the odds of capturing the lesion in the imaging plane, and appears to allow for the progress of lesion growth to be monitored accurately. Future studies will include ARFI beam sequence modifications (such as reducing the number of tracking beams used at each location and ECG triggering) that will both decrease the ARFI data acquisition time to far less than the current duration of 360 ms and increase the likelihood of completing an entire data acquisition while the heart is moving the least (i.e. during diastole).

The displacements shown in the ARFI images presented in FIGS. 8A-8J are approximately 2.5 times larger than those in FIGS. 7A-7D. The reason for this is twofold. First, the axial focal depth in second trial was 10 mm (as opposed to 15 mm in the first trial), meaning that the high intensity pushing beams experienced a smaller degree of near-field attenuation before reaching their target depth. Secondly, a longer pushing pulse length was used in the second trial (75 microseconds instead of 45 microseconds), meaning that larger intensities, and thus larger radiation forces, were applied to the tissue. A characteristic present in ARFI images is a focal gain in displacement located in the focal region of the pushing beam. As focal depths are typically chosen to correspond with structures being investigated, focal gains can be beneficial since in stiffer tissues stronger pushes enable the generation of displacements larger than the noise floor of the image. However, as lesions grow in size, portions of the lesion can lie outside of the focal region of the pushing beams. Although information concerning portions of the lesion outside of the focal region is contained in raw displacement data, oftentimes it cannot be displayed on a meaningful scale in the image. Several mechanisms are available to help account for this deficiency, including multiple transmit foci and time-gain compensation, both of which are currently used in B-Mode images of diagnostic ultrasound scanners. The requirement for an increased number of beams, and thus a corresponding increase in acquisition time, limits the utility of the multiple focal zone technique for cardiac applications. However, as shown in FIGS. 10A-10B, time-gain compensation techniques can be implemented post-acquisition with improved lesion visualization. The relative stiffness of tissues at all axial depths can be examined more effectively using this method.

During the (non- M-Mode) ARFI data aquisition in this study, 50 tracking beams were fired consecutively after each pushing beam at a PRF of 10.6 kHz. The images provided in this example were produced by analyzing the echoes from the sixth tracking beam at each pushing location, providing a snapshot in time of each tissue region's response to the radiation force impulse. In addition to viewing these snapshots, the entire temporal response of the tissue to the applied radiation force can also be viewed by processing all 50 tracking beams into one movie. Experience in our laboratory indicates that lesion boundaries, as well as other tissue mechanical properties, may be visualized effectively in this manner.

### EXAMPLE 2

The ability of ARFI imaging to monitor the ablation of soft tissues both ex vivo and in vivo was investigated. Thermal lesions were induced both in freshly excised bovine liver samples and in myocardialtissue of live sheep. While conventional sonography was unable to visualize induced lesions, ARFI imaging was capable of monitoring lesion size-and boundaries. Agreement was observed between lesion size in ARFI images and in results from pathology.

Imaging/Data Acquisition

Experiments were performed with a Siemens Antares scanner (Siemens Medical Solutions USA, Inc., Ultrasound Division, Issaquah, Wash.) that has been modified to provide users with the ability to specify acoustic beam sequences and intensities, as well as access raw radio frequency data. A Siemens VF10-5 linear array was used to acquire data. Beam sequences during ARFI data collection consisted of both tracking and pushing beams. The tracking beams were standard B-mode pulses (6.67 MHz center frequency, F/1.5 focal configuration, apodized, pulse repetition frequency (PRF) of 10.6 KHz, with a pulse length of 0.3 μs). The system utilizes dynamic focusing in receive such that a constant F/number of 1.5 is maintained. The pushing beam aperture was unapodized with a F/1.5 focal configuration, a center frequency of 6.67 MHz, and a pulse length range of 45-75 μs.

ARFI images were generated using 64-72 pushing locations at focal depths of 10, 15, or 20 mm. At each pushing location both tracking and pushing beams were fired along the same line of flight, as in typical A-line interrogation. The first beam fired was a tracking beam used as a reference to record initial tissue position. Next, a pushing beam was fired that generated the impulse of radiation force. Following the removal of the radiation force, 50 tracking beams were fired to allow for the measurement of the temporal response of the tissue. Data was processed by performing 1-D cross-correlation in the axial dimension between sequentially acquired tracking lines. Each tracking line was divided into a series of search regions, and the location of the peak in the cross-correlation function between a 0.25 mm kernel in the first tracking line and a search region in the next tracking line was used to estimate axial tissue displacement in that region. The kernel regions overlapped one another by 75%.

In the in vivo study, a 2 ms linear motion filter was applied to raw RF echo data prior to off-line processing to remove artifacts stemming from cardiac motion.

Experimental Setup and Procedure

The ex vivo experiment involved the use of fresh bovine liver samples obtained from a butcher. Liver was soaked in degassed water to remove air pockets. A thin layer of plastic film was placed tightly over the liver sample and attached to the sound-absorbing resting pad in order to mechanically stabilize the sample. An RF-ablation system (Model 8002, Cardiac Pathways Corporation, Sunnyvale, Calif.) with a French catheter was used to induce lesions in the sample. The ablation catheter was inserted through the top of the water tank into the liver sample parallel to the elevation plane of the image. After confirming the desired placement of the catheter, it was raised in the elevation direction such that it was not visualized in either the B-mode or the ARFI images. A reference data series was then acquired. The catheter was then re-lowered into the imaging plane of the transducer, approximately 2 cm deep in the liver sample. An ablation procedure was performed by applying 17 Watts of power to the tissue for 60 seconds, which resulted in a peak tissue temperature of roughly 85° C. Upon completion of ablation, the catheter was returned to its reference position (marked reference lines on the catheter stem allowed for it to be accurately returned to its original position). The plastic film holding the liver to the resting pad was sufficiently taut to ensure that the sample did not move during this process. A second B-mode and ARFI data series was then obtained. This process was repeated for a second RF-ablation session, and a third data series was acquired.

In the in vivo study, two sheep were used as approved by the Institutional Animal Care and Use Committee at Duke University conforming to the Research Animal Use Guidelines of the American Heart Association. The ablation catheter was inserted into the left ventricle via the femoral artery. Lesions were created in the lateral wall of the left ventricle using system power settings of 12-17 W and ablation durations of 50-70 s. Temperatures at the tissue catheter interface were not available due to limitations imposed by the ablation system.

Experiments were performed with a hand-held transducer placed directly on the beating heart of the sheep through an open chest. During the first sheep experiment, B-Mode and ARFI images of regions of interest were acquired both before and after ablations had occurred. During the second sheep experiment, B-Mode and ARFI images were acquired before, during, and after ablation procedures.

Results

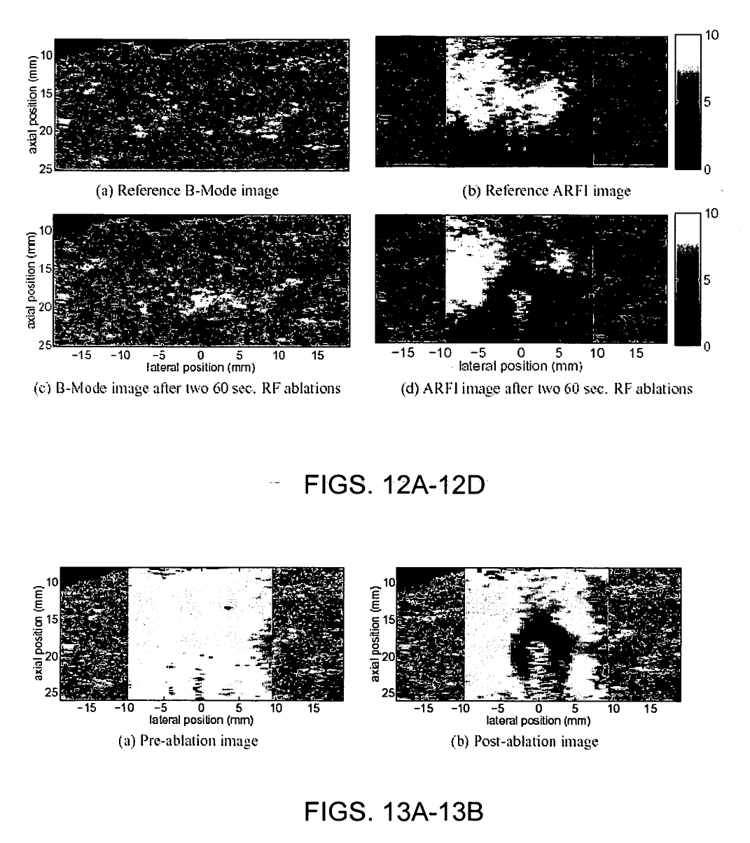

Results from the ex vivo experiment are shown in FIGS. 12A-12D. B Mode images of the liver sample are shown before (FIG. 12A) and after (FIG. 12C) two separate, 60 second RF ablation procedures were performed. It is evident that the thermal lesion created during the ablation process is not easily recognized in conventional B-Mode images. Although a large hyperechoic region, arising from gas bubble formation during heating, is present after two ablations (FIG. 12C), it does not provide reliable information concerning the size or location of the lesion.

ARFI images centered in the corresponding B-Mode images are shown in FIGS. 12B and 12D. The reference image shows that the liver sample initially exhibits fairly uniform stiffness across the region of interest. However, a sizeable region of stiff tissue (10 mm in diameter), corresponding to the induced thermal lesion, is apparent after the ablation procedures have been performed. The postablation ARFI image also exhibits noisy regions in the center of the lesion due to the presence of gas bubbles formed by tissue vaporization. While proximal and lateral lesion boundaries are well-defined, the distal lesion boundary is unseen. This lack of distal boundary definition is a result of the 20 mm focal depth of the image, as significantly less radiation force is applied beyond this axial position. A consequence of this is that although information concerning mechanical properties of tissue regions outside of the focal zone of the transducer are contained in raw data, they may be difficult to display on a meaningful scale in the image. To compensate for this deficiency, time-gain compensation (TGC) based processing techniques, commonly used in B-Mode images, can be applied to raw ARFI data. The results of exemplary TGC processing are shown in FIGS. 13A-13B.

As shown in FIGS. 13A-13B, utilization of TGC processing techniques allows for improved visualization of the distal lesion boundary. Although the color scale for the image no longer corresponds to actual tissue displacements, the relative stiffness of tissues at all axial depth scan now be examined more effectively. In cases where knowledge of lesion size and location are of paramount importance, TGC processing techniques may prove to be beneficial.

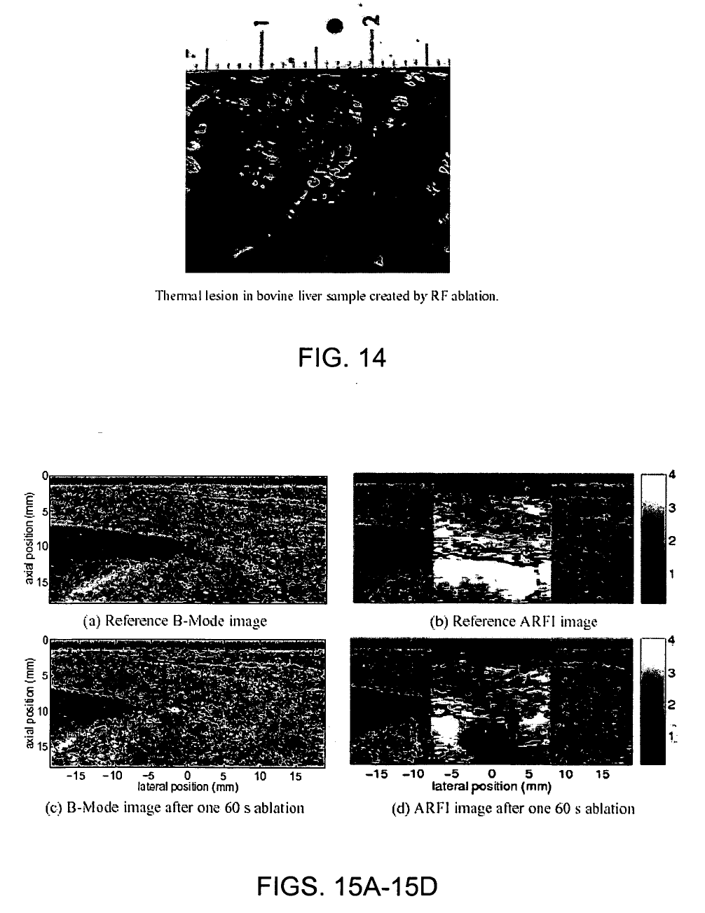

Upon completion of the ablation procedure, the liver sample was sliced in the approximate imaging plane of the transducer and examined. A palpable lesion (shown in the photograph provided in FIG. 14) was discovered where the tip of the RF catheter had been. Qualitative comparisons demonstrate good agreement between actual lesion size and size as shown in both the conventional and TGC processed ARFI images.

Results from the first sheep experiment are presented in FIGS. 15A-15D. FIGS. 15C and 15C show B-Mode images of the left ventricle's lateral wall before and after ablation, respectively. It is apparent from these images that conventional sonography displays little change in the cardiac tissue after a lesion has been induced, and that the presence of the lesion cannot even be verified in this instance.

FIGS. 15B and 15D show ARFI images centered in the corresponding B-Mode images. The reference ARFI image, shown in FIG. 15B, demonstrates that the cardiac tissue initially exhibits roughly uniform stiffness across the region under investigation. The effect of the 15 mm focal depth can also be observed as a region of increased displacement (>5 μm) ranging from 12-17 mm axially. The regions of low displacement (dark) located above 5 mm axially result from a reduced application of radiation force, not necessarily from an increase in tissue stiffness.

After one 60 second ablation procedure, a second ARFI image was acquired (FIG. 15D). Due to cardiac motion, the region of tissue located between 3 and 8 mm laterally in FIG. 15B is now located approximately between −3 and 2 mm laterally. FIGS. 15A-15D demonstrates that the ablated region has experienced an increase in stiffness relative to untreated tissue, and thus the resulting lesion is visualized well utilizing radiation force techniques. The lesion cross-section is circular in shape, with a diameter of approximately 5 mm. The proximal and lateral lesion boundaries are clearly distinguished, and regions of treated and untreated tissue are easily discerned. The distal lesion boundary lies beyond the focal zone of the ARFI image, and is thus not easily recognized without the implementation of TGC processing techniques (see FIG. 17).

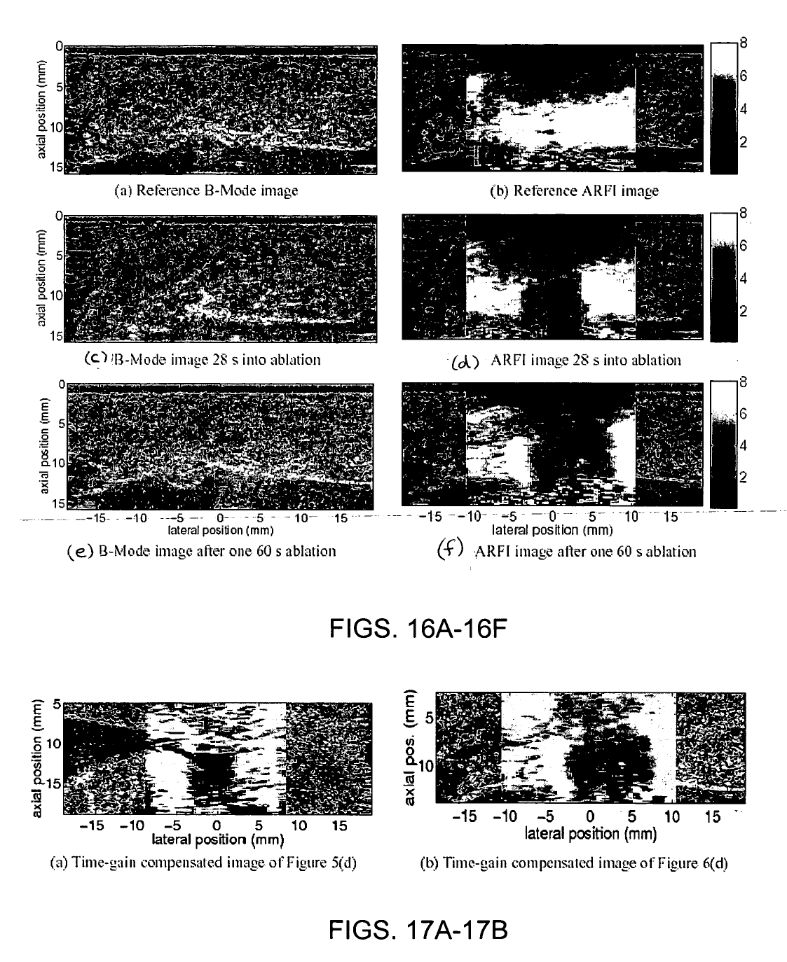

The results from the second sheep trial are shown in FIGS. 16A-16D. During this experiment, B-Mode and ARFI images were acquired before, during (every seven seconds), and after a 60 ablation procedure was performed. Due to space considerations, only images acquired prior to, 28 seconds into, and post-ablation are presented. The results are similar to those presented in FIGS. 15A-15D. The B-Mode images show minimal, if any change, throughout the course of the ablation procedure. However, the ARFI images show the lesion gradually growing in size as the ablation is performed. The distal lesion boundary lies on the endocardium, accounting for the lesion's asymmetric shape as it ends abruptly at the tissue-blood interface. The remaining lesion boundaries are visualized well until the post ablation image, where the lesion has grown large enough to include tissue regions outside the focal region of the image. As shown in FIG. 17, TGC processing allows for all lesion boundaries to be visualized well.

Discussion

It has been demonstrated that ARFI imaging is capable of detecting cardiac tissue mechanical properties in vivo. ARFI imaging was used to investigate a beating heart. From the resulting data anatomical features can be visualized, such as blood-tissue interfaces, as well as view both the spatial and temporal responses of the myocardium to the applied acoustic radiation force. Initial indications suggest that ARFI can be used safely to investigate a living heart, as no arrhythmias were detected in the ECG of the animal at any point during data acquisition. Although in our experiment the transducer was placed directly on the heart through an open chest, more clincally-realistic procedures can be adapted to easily by using a phased array to transmit transcutaneously through the rib cage.

The results presented indicate that, although conventional sonography fails to visualize induced thermal lesions, ARFI imaging is a promising modality for monitoring RF ablation therapy in vivo. The ability of ARFI imaging to distinguish lesions effectively stems from the large increase in their elastic modulus relative to untreated tissue. As a result, lesions are affected little in comparison to healthy tissues by applied radiation forces, and thus show up as regions of relatively smaller displacements in ARFI images. As conventional sonography is often used to guide ablation catheters, ARFI imaging may be a convenient modality for monitoring lesion formation.

### EXAMPLE 3

The ability of acoustic radiation force impulse (ARFI) imaging to visualize thermally- and chemically-induced lesions in soft tissues was investigated. Lesions were induced in freshly excised bovine liver samples. Chemical lesions were induced via the injection of formaldehyde, and thermal lesions were created using a radiofrequency ablation system. While conventional sonography was unable to visualize induced lesions, ARFI imaging was capable of monitoring lesion size and boundaries. Agreement was observed between lesion size in ARFI images and in results from pathology. ARFI imaging may be a promising modality for monitoring lesion development in situations where sonography is already involved as a guiding mechanism, such as in procedures requiring precise catheter placement.

Experiments were performed with a Siemens Anteres scanner (Siemens Medical Solutions USA, Inc., Ultrasound Division, Issaquah, Wash.) that has been modified to provide users with the ability to specify acoustic beam sequences and intensities. In addition, the machine has been altered such that users are capable of accessing raw radiofrequency data. A Siemens VF10-5 linear array was used to acquire data. This array consists of 192 elements, each 5 mm tall and approximately 0.2 mm wide. A fixed-focus acoustic lens is used in the elevation direction, while focusing in the lateral dimension is achieved electronically via the application of appropriate delays to each active element.

Beam sequences during the ARFI data collection consisted of both tracking and pushing beams. The tracking beams were standard B-Mode pulses (6.67 MHz center frequency, F/2 focal configuration, apodized, pulse repetition frequency (PRF) of 10.6 kHz, with a pulse length of 0.3 μs). The system utilizes dynamic focusing in receive such that a constant F/number of 2 is maintained. The beamwidth of the tracking beam can be calculated as λ*F/number, or 0.46 mm. The pushing beam aperture was unapodized with a F/1.5 focal configuration, a center frequency of 6.67 MHz, and a pulse length of 45 oe s. The shape of the focal region of the pushing beams is oblong (approximately 4 mm axially, and 0.45 mm laterally and in elevation) and fairly complex. Echoes from pushing pulses were not processed.

ARFI images were generated using 54 pushing locations at a focal depth of 20 mm. Pushing locations were separated laterally by a distance of 0.28 mm, resulting in a lateral region of interest (ROI) of 15.4 mm. At each pushing location both tracking and pushing beams were fired along the same line of flight, as in typical A-line interrogation. The first beam fired was a tracking beam used as a reference to record initial tissue position. Next, a pushing beam was fired that generated the impulse of radiation force. Following the removal of the radiation force, 50 tracking beams were fired to allow for the measurement of the temporal response of the tissue. Each pushing location was imaged for 4.9 ms, allowing for data from all 54 pushing locations to be acquired in 265 ms.

Raw RF echo data were processed off-line by performing 1-D cross-correlation in the axial dimension between sequentially acquired tracking lines. Each tracking line was divided into a series of search regions, and the location of the peak in the cross-correlation function between a 0.25 mm kernel in the first tracking line and a search region in the next tracking line was used to estimate axial tissue displacement in that region. The kernel regions overlapped one another by 75%.

Experimental Setup and Procedure

Several trials of chemical and thermal ablation experiments were conducted. The chemical ablation procedures involved using formaldehyde as a cross-linking agent to create subdermally-located stiff inclusions in fresh bovine liver samples obtained from a butcher. Although ethanol would be a more clinically-relevant chemical agent than formaldehyde, most procedures involving ethanol injection treat carcinomas of sizes on the order of 3 cm with chemical volumes on the order of 6-25 ml, whereas a formaldehyde injection of significantly less volume could induce a lesion of similar size. An advantage of formaldehyde was thus the ability to create a lesion while injecting a minimal amount of fluid into the liver sample. In addition, our laboratory experience suggests that the fast-acting nature of formaldehyde allows for it to create lesions of more predictable size and shape when compared to similar trials involving ethanol injection. As the purpose of this study was to demonstrate the feasibility of using ARFI imaging to visualize necrotic tissue regions, the formaldehyde serves as a suitable substitution for ethanol.

The crosslinking ability of the aldehyde family, most notably glutaraldehyde, has been verified in previous studies. When aldehydes are introduced into collagenous tissues, exposed regions will exhibit an increased Young's modulus and an increased bending stiffness due to aldehyde fixation. The result is a localized stiffened region surrounded by unaffected tissue. Although glutaraldehyde is known to be the most efficient generator of chemically and thermally stable cross-links in the aldehyde family, formaldehyde was chosen for our experiment due to its relative ease of handling and use.

The liver samples used for the experiments were approximately 5 cm by 7 cm by 10 cm in size. Liver samples were soaked in degassed water at room temperature for roughly 5 hr to remove air within them. The samples were then placed into a windowed water tank. The tank was lined with a layer of sound-absorbing material in order to reduce unwanted echoes from its sides. A thin layer of plastic film was placed tightly over the liver and attached to the sound-absorbing resting pad in order to mechanically stabilize the sample. The transducer was placed against the acoustically-transparent window on the outside of the water tank. The geometry of the acoustic window caused for a water stand-off of approximately 5-10 mm to exist between the transducer face and the liver sample. A mechanical translation stage (NEWPORT Electronics, Inc., Santa Ana, Calif.) was used to hold a 1 ml syringe in a manner such that the plunger could be depressed without the syringe itself moving. The translation stage allowed for the syringe to be moved with excellent precision within the water tank through the tank's top entry.

To begin the experiment, the translation stage was used to insert the needle of the syringe into the liver sample. Normal B-Mode imaging was used as a guide to adjust needle insertion location until it was approximately laterally centered in the image. The syringe was then withdrawn 3 mm in the elevation direction, moving it just out of the field of view (FOV) of the B-Mode image. Reference B-Mode and ARFI images were then obtained. The plunger of the syringe was then slowly depressed, injecting 0.4 ml of formaldehyde into the liver sample. Subsequent series of BMode and ARFI raw data were collected at 2 min intervals, beginning with 2 min after injection and ending 10 min after injection.

The second set of experiments involved the use of a RF-ablation system (Model 8002, Cardiac Pathways Corporation, Sunnyvale, Calif.) to create thermal lesions in fresh bovine liver samples. The liver samples used for the experiment were approximately 5 cm by 7 cm by 10 cm in size. The experimental setup was similar to that used in the chemical lesion experiment with the only deviation being the removal of the mechanical translation stage. The ground clip of the ablation system was attached to the sound absorbing material in the back corner of the water tank. A small amount of saline was added to the degassed water contained in the tank such that the impedance between the ablation catheter and the ground clip would fall to within the safety limits imposed by the ablation system. A 10 French catheter was inserted through the top of the water tank into the liver sample parallel to the elevation plane of the image. Conventional sonography was used to guide the catheter insertion location, which was approximately centered laterally. After confirming the desired placement of the catheter, it was raised in the elevation direction such that it was not visualized in either the B-Mode or the ARFI images. A reference B-Mode and ARFI data series was then taken with the catheter well out of the imaging plane. The catheter was then lowered into the imaging plane of the transducer, approximately 2 cm deep in the liver sample. Ablation procedures were then performed by applying 12-17 W of power to the tissue for durations ranging from 40-60 s. Upon completion of ablation, the catheter was returned to its reference position (marked interations on the catheter stem allowed for it to be accurately returned to its original position). The plastic film holding the liver to the resting pad was sufficiently taut to ensure that the sample did not move during this process. A second B-Mode and ARFI data series was then obtained. This process was repeated for a second RF-ablation session, and a third data series was acquired. To investigate the possibility of tissue temperature affecting displacements, the sample was allowed to cool for five min, and a fourth data set was captured. Following data acquisition, additional lesions were induced in the liver samples with the same ablation settings used in the actual experiment, but now with thermocouples inserted at the ablation site to monitor tissue temperatures. Thermocouple readings indicated that peak tissue temperatures at the lesion centers ranged from 85-100° C.

To conclude both the chemical and thermal lesion experiments, the liver samples was examined to measure lesion formation. Samples were sliced through the approximated imaging plane of the transducer with a butcher knife and inspected visually. Lesion sizes were measured by hand using an ordinary ruler, and photographs were taken to record resulting lesion size and shape.

Results Chemically-Induced Lesion Experiment

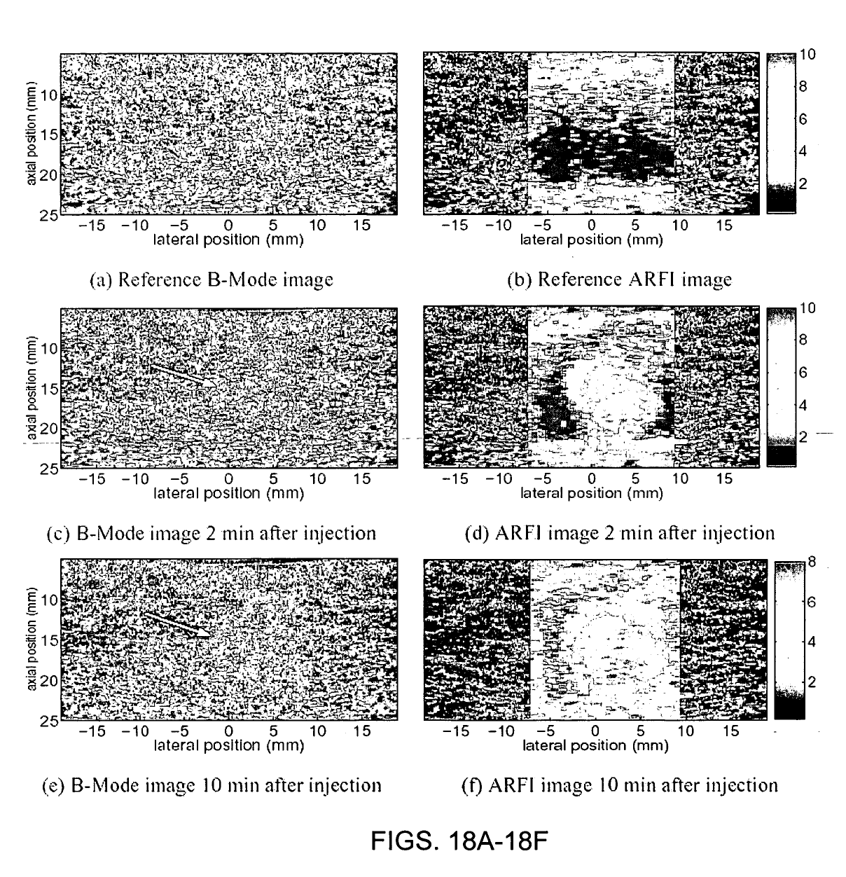

B-Mode and ARFI images of the bovine liver sample acquired before and after formaldehyde injection are shown in FIGS. 18A-18F. FIG. 18A shows the B-Mode reference image taken prior to chemical injection, while FIGS. 18C and 18E show B-Mode images taken 2 and 10 min after formaldehyde injection, respectively. FIGS. 18B, 18D and 18F show the ARFI images centered in the corresponding B-Mode images. For the results presented, the ARFI images were intentionally acquired off-center from the corresponding B-Mode images in order to position the injection point in the center of the displacement maps.

FIG. 18C shows that 2 min after chemical injection, a hyperechoic region (with respect to the reference image) exists that corresponds to the fluid introduced into the liver. This hyperechoic region is also present in the B-Mode image taken after 10 min of chemical exposure (FIG. 18E). However, after 10 min have elapsed the hyperechoic region is beginning to wane, likely due to the resorption of any air bubbles introduced during injection and the diffusion of formaldehyde away from the injection point. In both of these conventional B-Mode images, it is difficult to discern lesion boundaries, as well as overall lesion shape.

The reference ARFI image, shown in FIG. 18B demonstrates that the liver sample initially exhibits roughly uniform stiffness across the region under investigation. The effect of the 20 mm axial focal depth can be observed as a region of increased displacement ranging from 12-22 mm. The regions of low displacement (blue) located deeper than 22 mm axially result from a reduced application of radiation force, not from an increase in tissue stiffness.

FIG. 18D shows that after 2 min of chemical exposure, an elliptical region of increased stiffness (characterized by a decreased displacement) is present in the imaging plane. In this image, the boundaries between the lesion and untreated tissue are clearly distinguished. FIG. 18F displays the ARFI image taken after 10 min of formaldehyde exposure. The image indicates that the diffusion of the chemical agent has caused the lesion to both grow in size and change slightly in overall shape. As in the previous image, the boundary between the lesion and the surrounding healthy tissue is clearly defined in the ARFI displacement map. One will notice that displacements in FIG. 18F are slightly smaller (peaks of roughly 8 μm as opposed to 10 μm) than in the previous images. This is due to the fact that the axial focus in this image was changed to 25 mm in order to ensure enough radiation force was applied at this depth to visualize the distal lesion boundary. As focal depths are increased, more output power must be utilized to achieve similar pushing strengths. Since the current system settings provided the radiation force strengths necessary to visualize the lesion, power supply configurations were not altered.

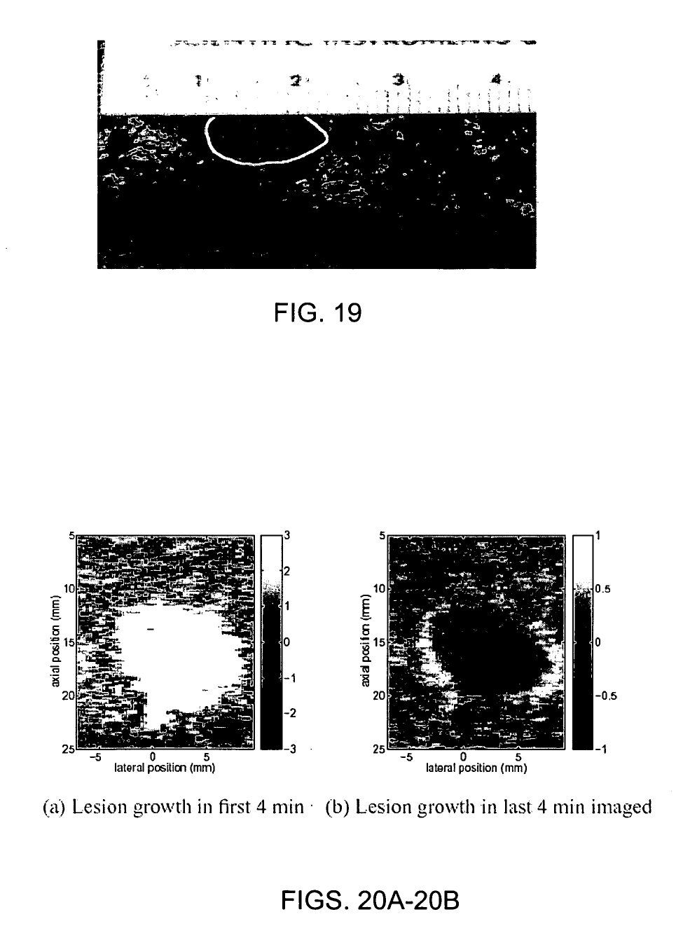

A photograph of the formaldehyde-induced lesion (outlined in white) obtained 15 min after chemical injection is shown in FIG. 19. Our laboratory experience suggests that formaldehyde-induced lesions created with small chemical volumes (<0.5 ml) stop growing significantly in size after approximately 8 min of chemical exposure, and thus we would not expect lesion size after 15 min to differ appreciably from when it was last imaged (after 10 min of exposure). (FIGS. 20A-20B illustrate the increasing stability of the lesion by comparing lesion growth in the first 4 min of formaldehyde exposure (FIG. 20A with lesion growth during the last 4 min it was imaged (FIG. 20B).) Measurement of the lesion cross-section showed a maximum diameter of roughly 12 mm, which corresponds well to the diameter of the same region (roughly 11.5 mm) in the ARFI image of FIG. 18F. The shape of the lesion presented in the photograph also verifies the shape of the lesion as depicted in the ARFI image, with the right side of the region exhibiting a sharper curvature than the left side.

Thermal Lesion Experiment

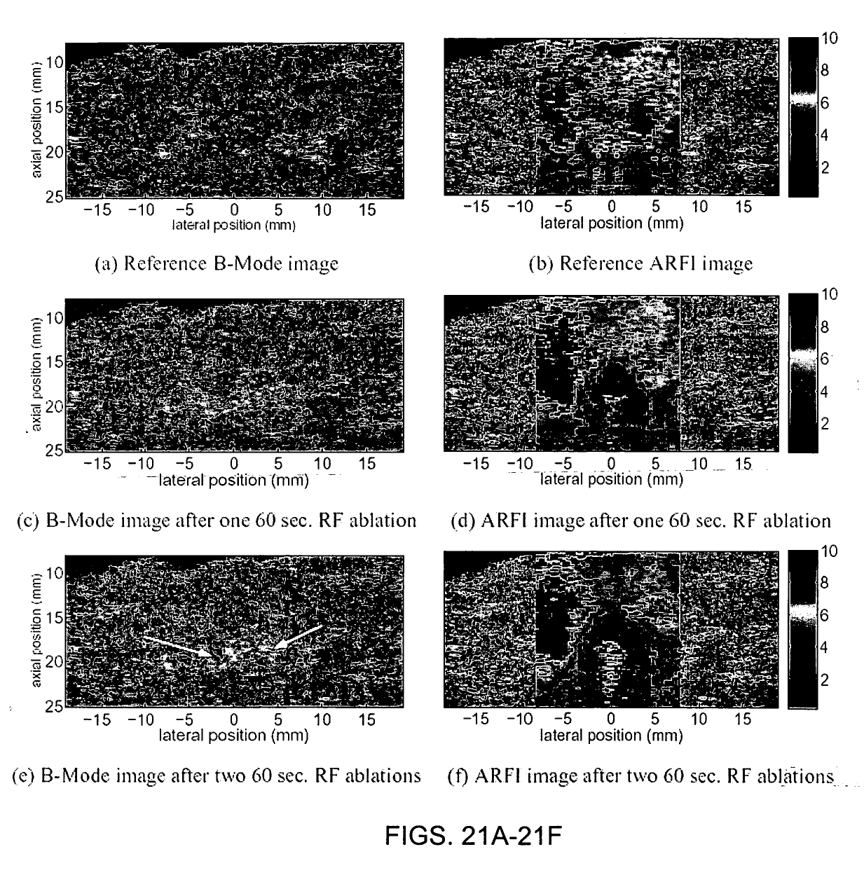

The B-Mode and ARFI images of the liver sample acquired during the RF ablation experiment are shown in FIGS. 21A-21F. The ablation site is located at a 20 mm axial depth and is approximately centered laterally. It is evident that the thermal lesion created during the ablation process is not easily recognized during conventional B-Mode imaging. Although a large hyperechoic region, arising from gas bubble formation during heating, is present after two ablations (FIG. 21E, it does not provide reliable information concerning the size or location of the lesion.

The ARFI images centered in the corresponding B-Mode images are shown in FIGS. 21B, 21D and 21F. The focal depth effects present in the chemical lesion experiment are again apparent here, and no significant radiation force is applied at axial depths beyond roughly 22 mm. The reference image (FIG. 21B) shows that the untreated liver sample exhibits fairly uniform stiffness across the region of interest. However, a sizeable region of stiff tissue (8 mm in diameter), corresponding to the induced thermal lesion, is apparent after one 60 s RF ablation (FIG. 21D). After a second, identical ablation procedure, the size of the detected thermal lesion grows slightly larger (10 mm diameter, FIG. 21F). In both cases, boundaries of the thermal lesion are well-defined, and lesion size is easily determined. The exception is the distal lesion boundary, which is not visualized in the images. The lack of distal boundary definition arises from the reduced application of radiation force at this depth, and is thus a direct consequence of the focal depth chosen for the images. The postablation ARFI images also exhibit noisy regions in the center of the lesion due to the presence of gas bubbles formed by tissue vaporization (most pronounced after two ablations, FIG. 21F). These vapor pockets are formed in tissue regions adjacent to the tip of the catheter, where temperatures are the highest. The ARFI image acquired 5 min after the second ablation process is virtually identical to the image in FIG. 21F, and is thus not presented here.

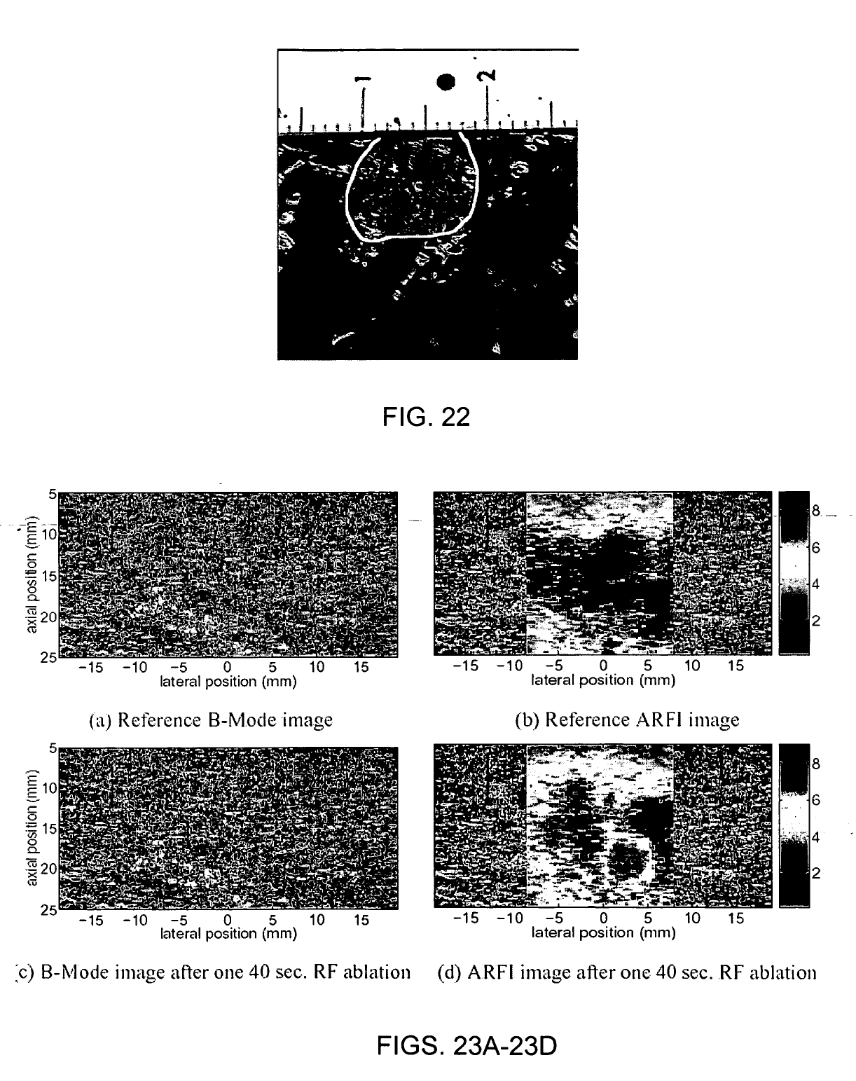

Upon completion of imaging the RF ablation process, the liver sample was sliced and examined to confirm that a thermal lesion was created. A palpable lesion was discovered in the location where the tip of the RF catheter had been. A photograph of this lesion (outlined in white) is shown in FIG. 22. Estimations of actual maximum lesion diameter (roughly 10 mm) were again in relatively good agreement with the diameter of the lesion indicated in the ARFI image (roughly 9 mm). As the slice through the liver sample was merely approximated as being the imaging plane of the transducer, precise quantitative comparisons of actual lesion size and lesion size in ARFI images cannot be made with confidence.

As mentioned, the distal boundary of the lesion depicted in FIGS. 21A-21F was located beyond the axial focus of the lesion, where little radiation force is applied, and was thus not visualized. FIGS. 23A-23D shows an B-Mode and ARFI images acquired at a deeper focus (25 mm) of a lesion induced in a different liver sample. This lesion was created using low energy ablation system settings (12 W for 40 s) in order ensure that it would not grow in size beyond the focus of the image. These reduced power settings kept tissue temperatures at lower levels than in previous ablations, and thus there is no evidence in the post-ablation B-Mode image (FIG. 23C) of a hyperechoic region corresponding to tissue vaporization. As shown in the post-ablation ARFI image (FIG. 23D), lesion location and shape are once again visualized, and now all lesion boundaries can be easily recognized.

Discussion and Conclusions

The images provided in this example were created using a transmit frequency of 6.67 MHz, the center frequency of the transducer array used in the experiment. Although this is a frequency that would be appropriate for use with many intra-operative procedures, non-invasive clinical monitoring of lesion development in the liver would typically require an array transmitting transcutaneously at frequency range of 3-5 MHz. As experiments were performed ex vivo in a water tank, the chosen transmit frequency of 6.67 MHz was appropriate for our purposes. Current work is being performed to investigate the possibility of using lower transmit frequencies to supply radiation force more efficiently to deeper-lying tissues.

In the cases presented there exists good agreement between lesions visualized in ARFI images and the results from pathology. Comparisons of lesion size, as measured by maximum lesion diameter in the lateral plane, between ARFI images and actual lesion cross-sections through the approximated imaging plane showed agreement to within 10% error. Also, lesion shapes in the ARFI images corresponded directly to actual lesion cross-sections, as indicated by the photographs provided in FIGS. 19 and 22. These results are consistent with others (not presented herein) from similar induced-lesion experiments performed in our laboratory, where qualitative inspection and approximated measurements suggest that ARFI images accurately reflect lesion size and shape. However, challenges in slicing liver samples exactly in the imaging plane of the transducer during examination limits the precision of size comparisons, and thus a future study which makes accurate, quantitative assessments of ARFI imaging's ability to determine lesion size would be beneficial.

The ability of ARFI imaging to clearly distinguish lesions from surrounding healthy tissues arises from the large increase in elastic modulus associated with the lesion. Assuming a uniform distribution of radiation force, the lesion would be displaced much less in response to the force than a healthy, less stiff region of tissue. For the results presented here, healthy tissue displacements were in the range of 6-10 μm, while lesions were typically displaced 1-2 μm. The assumption of uniform radiation force distribution, however, may be invalid, as the attenuation coefficient of tissue may be permanently increased when tissue temperatures are raised above 40 ° C. (the result of irreversible structural changes caused by coagulation). The applied radiation force is proportional to the tissue attenuation, and therefore it is likely that a stronger radiation force was applied to the lesion than was applied to the healthy tissue. Thus, the actual change in the elastic modulus of the lesions during their formation may be greater than as demonstrated in the images. It has also been noted that at these elevated temperatures, structural effects on tissue attenuation coefficient dominate any attenuation changes that may occur due to heating. This is consistent with the fact that ARFI images acquired immediately after an RF ablation are virtually identical to images acquired 5 min after an RF ablation, even though significant cooling has occurred.

During data aquisition, 50 tracking beams are fired consecutively after each pushing beam at a PRF of 10.6 kHz. The ARFI images provided in this study were produced by analyzing the echoes from the eighth tracking beam at each pushing location, providing a snapshot in time of each tissue region's response to the radiation force impulse. However, it is often beneficial to view the entire transient response for the region of interest. By processing the results from all 50 tracking beams into one movie, the temporal response of the tissue to the applied radiation force can be viewed over a 4.7 ms window.

In addition to images of tissue displacement, ARFI imaging is capable of producing images of other tissue characteristics, such as maximum displacement, time needed to reach peak displacement, and recovery velocity (i.e., the slope of the displacement/time curve as the tissue recovers to its initial position). Each of these alternative image types has been previously shown to provide valuable information concerning the mechanical properties of tissue under investigation, and in certain cases they may be more desirable than conventional images of displacement.

It has been demonstrated that ARFI imaging is capable of detecting formaldehyde- and thermally induced soft tissue lesions that conventional sonography may be unable to visualize. ARFI imaging may be a promising modality for monitoring thermal lesion development in situations, where sonography is already involved as a guiding mechanism, such as in many procedures requiring precise catheter placement. Its low cost and portability give ARFI imaging a distinct advantage over MR methods for this purpose.

### EXAMPLE 4

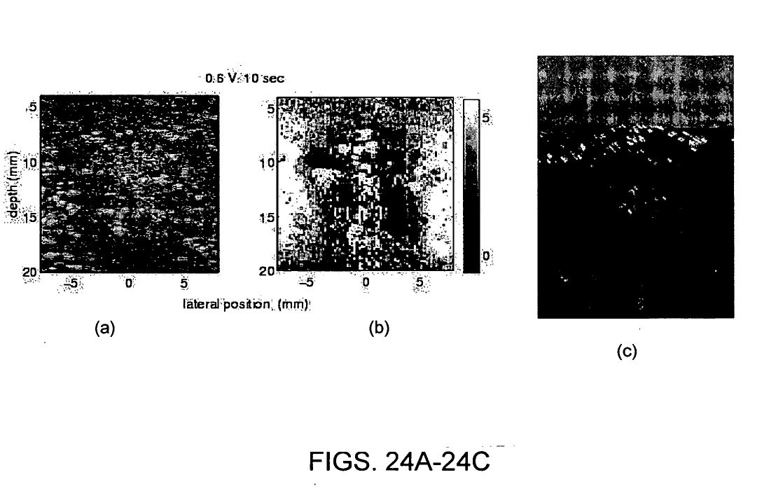

FIGS. 24A-24C illustrate images of an ex vivo bovine liver sample after HIFU ablation. FIG. 24A is a conventional B-Mode ultrasound image, FIG. 24B is an ARFI displacement image, and FIG. 24C is a pathology image of the ex vivo bovine liver sample after HIFU ablation. The HIFU system used a 1 MHz, piston transducer transmitting continuous wave ultrasound for 10 seconds in order to generate the ablation lesion of FIGS. 24A-24C. As shown in FIG. 24A, this resulted in the bubble formation in the B-mode image as shown by the bright white regions, and a slight enhancement in echo signal from the center of the ablated region. However, the extent of the bubbles and B-mode signal enhancement do not accurately portray the lateral extent of the ablation lesion. In the ARFI displacement image of FIG. 24B, a darkened region (e.g. stiffer region) corresponds to the size of the ablation region. As can be seen in FIGS. 24A and 24B, the air bubbles shown in the B-mode image (FIG. 24A) generate regions of increased displacement (white spots) within the ablation lesion in the ARFI image (FIG. 24B). However, the presence of the air bubbles does not obscure the lateral lesion margins in this image. In the drawings and specification, there have been disclosed specific embodiments of the invention and, although specific terms are employed, they are used in a generic and descriptive sense only and not for purposes of limitation, the scope of the invention being set forth in the following claims.

1. An ultrasound method of distinguishing ablated tissue from unablated tissue, the method comprising:
scanning ablated tissue using Acoustic Radiation Force Impulse (ARFI) imaging; and
generating ARFI image data based on the scanning, the image data including a portion thereof corresponding to a region of increased stiffness representing the ablated tissue that is distinguishable from another portion of the image data corresponding to unablated tissue.

2. The method of claim 1, wherein scanning the tissue using ARFI comprises:
delivering a tracking pulse from an ultrasound transducer array to the tissue to detect an initial position for the tissue;
delivering a pushing pulse from the ultrasound transducer array to the tissue to displace the tissue to a displaced position; and
delivering a second tracking pulse from the ultrasound transducer array to the tissue to detect the displaced position of the tissue.

3. The method of claim 1, wherein the scanning is performed by an ultrasound transducer array on a catheter.

4. The method of claim 1, wherein the scanning is performed by an ultrasound transducer array on an endoscope.

5. The method of claim 1, further comprising ablating tissue to provide the ablated tissue.

6. The method of claim 5, further comprising identifying characteristics of the ablated tissue using the ARFI image.

7. The method of claim 6, wherein the characteristics of the ablated tissue include the size of the ablated portion and/or the position of the ablated tissue.

8. The method of claim 5, further comprising repeating the scanning step while ablating the tissue to monitor a size and/or shape of the ablated tissue.

9. The method of claim 8, further comprising controlling ablating the tissue based on the ARFI image.

10. The method of claim 5, wherein the ablating is performed by an ablation element on a catheter.

11. The method of claim 10, wherein the scanning is performed by an ultrasound transducer array on the catheter.

12. The method of claim 5, wherein the ablating is performed by an ablation element on an endoscope.

13. The method of claim 12, wherein the scanning is performed by an ultrasound transducer array on the endoscope.

14. The method of claim 1, wherein the ARFI image comprises a three dimensional ultrasound image.

15. The method of claim 1, wherein the ARFI image comprises a first ARFI image, the method further comprising:
subsequently scanning the tissue using ARFI imaging to provide a second ARFI image;
comparing the first ARFI image and the second ARFI image; and
determining whether a change has occurred in the ablation portion of the tissue based on the comparison.

16. A computer program product for distinguishing ablated tissue from unablated tissue, the computer program product comprising:
a computer readable medium having computer readable program code embodied therein, the computer readable program code comprising:
computer readable program code configured to scan ablated tissue using Acoustic Radiation Force Impulse (ARFI) imaging; and
computer readable program code configured to generate ARFI image data based on the scanning, the image data including a portion thereof corresponding to a region of increased stiffness representing the ablated tissue that is distinguishable from another portion of the image data corresponding to unablated tissue.

17. The computer program product of claim 16, wherein the computer readable program code configured to scan ablated tissue comprises:
computer readable program code configured to deliver a tracking pulse from an ultrasound transducer array to the tissue to detect an initial position for the tissue;
computer readable program code configured to deliver a pushing pulse from the ultrasound transducer array to the tissue to displace the tissue to a displaced position; and
computer readable program code configured to deliver a second tracking pulse from the ultrasound transducer array to the tissue to detect the displaced position of the tissue.

18. An ultrasound system for distinguishing ablated tissue from unablated tissue, the system comprising:
an ultrasound transducer array configured to scan ablated tissue using Acoustic Radiation Force Impulse (ARFI) imaging; and
a processor configured to generate ARFI image data based on the scanning, the image data including a portion thereof corresponding to a region of increased stiffness representing the ablated tissue that is distinguishable from another portion of the image data corresponding to unablated tissue.

19. The ultrasound system of claim 18, wherein the processor is configured to scan the ablated tissue by delivering a tracking pulse from the ultrasound transducer array to the tissue to detect an initial position for the tissue; delivering a pushing pulse from the ultrasound transducer array to the tissue to displace the tissue to a displaced position; and delivering a second tracking pulse from the ultrasound transducer array to the tissue to detect the displaced position of the tissue.

20. The ultrasound system of claim 17, further comprising an ablation element configured to ablate the ablated tissue.

21. The ultrasound system of claim 20, further comprising a catheter, wherein the ultrasound transducer array and the ablation element are positioned on the catheter.

22. The ultrasound system of claim 20, further comprising an endoscope, wherein the ultrasound transducer array and the ablation element are positioned on the endoscope.

23. A method of ablating tissue, the method comprising:
ablating a portion of the tissue;
scanning the tissue using Acoustic Radiation Force Impulse (ARFI) imaging to provide an ARFI image of the ablated portion of the tissue; and
identifying characteristics of the ablated portion of the tissue using the ARFI image.

24. The method of claim 23, wherein ablating the portion of the tissue is performed by an ablation element and scanning the tissue is performed by an ultrasound transducer array, the ablation element and transducer array being position on a catheter.

25. The method of claim 23, wherein ablating the portion of the tissue is performed by an ablation element and scanning the tissue is performed by an ultrasound transducer array, the ablation element and transducer array being position on an endoscope.

26. A device comprising:
a transducer delivery device; and
an ultrasound transducer array on the delivery device configured to scan ablated tissue using Acoustic Radiation Force Impulse (ARFI) imaging, the transducer delivery device being configured to deliver the ultrasound transducer array to ablated tissue inside a patient.

27. The device of claim 26, wherein the delivery device comprises a catheter.

28. The device of claim 26, wherein the delivery device comprises an endoscope.

29. The device of claim 26, further comprising an ablation element on the deliver device configured to ablate tissue.

30. The device of claim 29, wherein the ablation element is an ultrasound ablation transducer element in the ultrasound transducer array that is configured to ultrasonically ablate tissue.

31. The device of claim 26, wherein the ultrasound transducer array comprises a plurality of transducer elements, the transducer elements being configured to ultrasonically ablate tissue and to scan the ablated tissue using ARFI imaging.
